SEQUENCE LISTING (100 sequences)

SEQ ID NO: 1 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctcgagaaggtatattgctgttgacagtgagcgctggagaagtttactacataaatagtgaagccacagatgtatttatgtagtaaacttctccattgcctactgcctcggaattcaaggggctactttaggagcaattatcttgtttactaaaactgaataccttgctatctctttgatacatttttacaaagctgaattaaaatggtataaattaaatcacttt

SEQ ID NO: 2 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcgacttcttaacccaacagaaggctcgagaaggtatattgctgttgacagtgagcgctggagaagtttactacataaatagtgaagccacagatgtatttatgtagtaaacttctccattgcctactgcctcggacttcaaggggctagaattcgagcaattatcttgtttactaaaactgaataccttgctatctctttgatacatttttacaaagctgaattaaaatggtataaattaaatcacttt

SEQ ID NO: 3 - DNA, 116 bp, Artificial Sequence
  Description of Artificial Sequence Synthetic polynucleotide
ctcgagaaggtatattgctgttgacagtgagcgaccagctaaaggtgaagatatatagtgaagccacagatgtatatatcttcacctttagctggctgcctactgcctcggaattc

SEQ ID NO: 4 - DNA, 116 bp, Artificial Sequence
  Description of Artificial Sequence Synthetic polynucleotide
ctcgagaaggtatattgctgttgacagtgagcgacagctaaaggtgaagatatattagtgaagccacagatgtaatatatcttcacctttagctggtgcctactgcctcggaattc

SEQ ID NO: 5 - DNA, 321 bp, Artificial Sequence
  Description of Artificial Sequence Synthetic polynucleotide
tgtttgaatgaggcttcagtactttacagaatcgttgcctgcacatcttggaaacacttgctgggattacttcttcaggttaacccaacagaaggctcgagaaggtatattgctgttgacagtgagcgactttatgtagtaaacttctccatgtgaagccacagatgatggagaagtttactacataaagctgcctactgcctcggaattcaaggggctactttaggagcaattatcttgtttactaaaactgaataccttgctatctctttgatacatttttacaaagctgaattaaaatggtataaattaaatcacttt

SEQ ID NO: 6 - DNA, 116 bp, Artificial Sequence
  Description of Artificial Sequence Synthetic polynucleotide
ctcgagaaggtatattgctgttgacagtgagcgactatatcttcacctttagctggcgtgaagccacagatggccagctaaaggtgaagatatagctgcctactgcctcggaattc

SEQ ID NO: 7 - DNA, 116 bp, Artificial Sequence
  Description of Artificial Sequence Synthetic polynucleotide
ctcgagaaggtatattgctgttgacagtgagcgacatatatcttcacctttagctgggtgaagccacagatgccagctaaaggtgaagatatatgctgcctactgcctcggaattc

SEQ ID NO: 8 - DNA, 590 bp, Artificial Sequence
  Description of Artificial Sequence Synthetic polynucleotide
cctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgactagggataacagggtaattgtttgaatgaggcttcagtactttacagaatcgttgcctgcacatcttggaaacacttgctgggattacttcttcaggttaacccaacagaaggctcgagcaaccagaattcaaggggctactttaggagcaattatcttgtttactaaaactgaataccttgctatctctttgatacatttttacaaagctgaattaaaatggtataaattaaatcacttttttcaattgacgcgtgatctaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgt

SEQ ID NO: 9 - DNA, 24 bp, Artificial Sequence
  Description of Artificial Sequence Synthetic primer
agaaggctcgagaaggtatattgc

SEQ ID NO: 10 - DNA, 30 bp, Artificial Sequence
  Description of Artificial Sequence Synthetic primer
gctcgaattctagccccttgaagtccgagg

SEQ ID NO: 11 - DNA, 116 bp, Artificial Sequence; identical to SEQ ID NO: 3
  Description of Artificial Sequence Synthetic polynucleotide

SEQ ID NO: 12 - DNA, 116 bp, Artificial Sequence; identical to SEQ ID NO: 4
  Description of Artificial Sequence Synthetic polynucleotide

SEQ ID NO: 13 - DNA, 116 bp, Artificial Sequence
  Description of Artificial Sequence Synthetic polynucleotide
ctcgagaaggtatattgctgttgacagtgagcgcaggaattataatgcttatctatagtgaagccacagatgtatagataagcattataattcctatgcctactgcctcggaattc

SEQ ID NO: 14 - DNA, 116 bp, Artificial Sequence
  Description of Artificial Sequence Synthetic polynucleotide
ctcgagaaggtatattgctgttgacagtgagcgccgacttagacttgacctatattagtgaagccacagatgtaatataggtcaagtctaagtcgatgcctactgcctcggaattc

SEQ ID NO: 15 - DNA, 116 bp, Artificial Sequence
  Description of Artificial Sequence Synthetic polynucleotide
ctcgagaaggtatattgctgttgacagtgagcgcttgggtaaatacgttcttcattagtgaagccacagatgtaatgaagaacgtatttacccaaatgcctactgcctcggaattc

SEQ ID NO: 16 - DNA, 116 bp, Artificial Sequence
  Description of Artificial Sequence Synthetic polynucleotide
ctcgagaaggtatattgctgttgacagtgagcgaaagatcagcattcacaaattatagtgaagccacagatgtataatttgtgaatgctgatcttctgcctactgcctcggaattc

SEQ ID NO: 17 - DNA, 116 bp, Artificial Sequence
  Description of Artificial Sequence Synthetic polynucleotide
ctcgagaaggtatattgctgttgacagtgagcgattatacaaggagacttctgaatagtgaagccacagatgtattcagaagtctccttgtataagtgcctactgcctcggaattc

SEQ ID NO: 18 - DNA, 116 bp, Artificial Sequence
  Description of Artificial Sequence Synthetic polynucleotide
ctcgagaaggtatattgctgttgacagtgagcgcaagggtaaacttgacagaagatagtgaagccacagatgtatcttctgtcaagtttacccttttgcctactgcctcggaattc

SEQ ID NO: 19 - DNA, 116 bp, Artificial Sequence
  Description of Artificial Sequence Synthetic polynucleotide
ctcgagaaggtatattgctgttgacagtgagcgcgcacaggaattttgtttaatatagtgaagccacagatgtatattaaacaaaattcctgtgcatgcctactgcctcggaattc

SEQ ID NO: 20 - DNA, 116 bp, Artificial Sequence
  Description of Artificial Sequence Synthetic polynucleotide
ctcgagaaggtatattgctgttgacagtgagcgcgactgatattaacaaagcttatagtgaagccacagatgtataagctttgttaatatcagtcttgcctactgcctcggaattc

SEQ ID NO: 21 - DNA, 116 bp, Artificial Sequence
  Description of Artificial Sequence Synthetic polynucleotide
ctcgagaaggtatattgctgttgacagtgagcgccaagtgttcggtgtaactaaatagtgaagccacagatgtatttagttacaccgaacacttgatgcctactgcctcggaattc

SEQ ID NO: 22 - DNA, 116 bp, Artificial Sequence
  Description of Artificial Sequence Synthetic polynucleotide
ctcgagaaggtatattgctgttgacagtgagcgactccggaaactggacattaaatagtgaagccacagatgtatttaatgtccagtttccggagctgcctactgcctcggaattc

SEQ ID NO: 23 - DNA, 116 bp, Artificial Sequence
  Description of Artificial Sequence Synthetic polynucleotide
ctcgagaaggtatattgctgttgacagtgagcgcggattgtgactcttatttctttagtgaagccacagatgtaaagaaataagagtcacaatccttgcctactgcctcggaattc

SEQ ID NO: 24 - DNA, 116 bp, Artificial Sequence
  Description of Artificial Sequence Synthetic polynucleotide
ctcgagaaggtatattgctgttgacagtgagcgaaagagtcactgtctgaatgaatagtgaagccacagatgtattcattcagacagtgactcttctgcctactgcctcggaattc

SEQ ID NO: 25 - DNA, 116 bp, Artificial Sequence
  Description of Artificial Sequence Synthetic polynucleotide
ctcgagaaggtatattgctgttgacagtgagcgaaacagcctcgatttttaagaatagtgaagccacagatgtattcttaaaaatcgaggctgttctgcctactgcctcggaattc

SEQ ID NO: 26 - DNA, 116 bp, Artificial Sequence
  Description of Artificial Sequence Synthetic polynucleotide
ctcgagaaggtatattgctgttgacagtgagcgcggactggttatagatttataatagtgaagccacagatgtattataaatctataaccagtccatgcctactgcctcggaattc

SEQ ID NO: 27 - DNA, 131 bp, Artificial Sequence
  Description of Artificial Sequence Synthetic polynucleotide
ctcgagaaggtatattgctgttgacagtgagcgaccagctaaaggtgaagatatatagtgaagccacagatgtatatatcttcacctttagctggctgcctactgcctcggacttcaaggggctagaattc

SEQ ID NO: 28 - DNA, 131 bp, Artificial Sequence
  Description of Artificial Sequence Synthetic polynucleotide
ctcgagaaggtatattgctgttgacagtgagcgacagctaaaggtgaagatatattagtgaagccacagatgtaatatatcttcacctttagctggtgcctactgcctcggacttcaaggggctagaattc

SEQ ID NO: 29 - DNA, 131 bp, Artificial Sequence
  Description of Artificial Sequence Synthetic polynucleotide
ctcgagaaggtatattgctgttgacagtgagcgcaggaattataatgcttatctatagtgaagccacagatgtatagataagcattataattcctatgcctactgcctcggacttcaaggggctagaattc

SEQ ID NO: 30 - DNA, 131 bp, Artificial Sequence
  Description of Artificial Sequence Synthetic polynucleotide
ctcgagaaggtatattgctgttgacagtgagcgccgacttagacttgacctatattagtgaagccacagatgtaatataggtcaagtctaagtcgatgcctactgcctcggacttcaaggggctagaattc

SEQ ID NO: 31 - DNA, 131 bp, Artificial Sequence
  Description of Artificial Sequence Synthetic polynucleotide
ctcgagaaggtatattgctgttgacagtgagcgcttgggtaaatacgttcttcattagtgaagccacagatgtaatgaagaacgtatttacccaaatgcctactgcctcggacttcaaggggctagaattc

SEQ ID NO: 32 - DNA, 131 bp, Artificial Sequence
  Description of Artificial Sequence Synthetic polynucleotide
ctcgagaaggtatattgctgttgacagtgagcgaaagatcagcattcacaaattatagtgaagccacagatgtataatttgtgaatgctgatcttctgcctactgcctcggacttcaaggggctagaattc

SEQ ID NO: 33 - DNA, 131 bp, Artificial Sequence
  Description of Artificial Sequence Synthetic polynucleotide
ctcgagaaggtatattgctgttgacagtgagcgattatacaaggagacttctgaatagtgaagccacagatgtattcagaagtctccttgtataagtgcctactgcctcggacttcaaggggctagaattc

SEQ ID NO: 34 - DNA, 131 bp, Artificial Sequence
  Description of Artificial Sequence Synthetic polynucleotide
ctcgagaaggtatattgctgttgacagtgagcgcaagggtaaacttgacagaagatagtgaagccacagatgtatcttctgtcaagtttacccttttgcctactgcctcggacttcaaggggctagaattc

SEQ ID NO: 35 - DNA, 131 bp, Artificial Sequence
  Description of Artificial Sequence Synthetic polynucleotide
ctcgagaaggtatattgctgttgacagtgagcgcgcacaggaattttgtttaatatagtgaagccacagatgtatattaaacaaaattcctgtgcatgcctactgcctcggacttcaaggggctagaattc

SEQ ID NO: 36 - DNA, 131 bp, Artificial Sequence
  Description of Artificial Sequence Synthetic polynucleotide
ctcgagaaggtatattgctgttgacagtgagcgcgactgatattaacaaagcttatagtgaagccacagatgtataagctttgttaatatcagtcttgcctactgcctcggacttcaaggggctagaattc

SEQ ID NO: 37 - DNA, 131 bp, Artificial Sequence
  Description of Artificial Sequence Synthetic polynucleotide
ctcgagaaggtatattgctgttgacagtgagcgccaagtgttcggtgtaactaaatagtgaagccacagatgtatttagttacaccgaacacttgatgcctactgcctcggacttcaaggggctagaattc

SEQ ID NO: 38 - DNA, 131 bp, Artificial Sequence
  Description of Artificial Sequence Synthetic polynucleotide
ctcgagaaggtatattgctgttgacagtgagcgactccggaaactggacattaaatagtgaagccacagatgtatttaatgtccagtttccggagctgcctactgcctcggacttcaaggggctagaattc

SEQ ID NO: 39 - DNA, 131 bp, Artificial Sequence
  Description of Artificial Sequence Synthetic polynucleotide
ctcgagaaggtatattgctgttgacagtgagcgcggattgtgactcttatttctttagtgaagccacagatgtaaagaaataagagtcacaatccttgcctactgcctcggacttcaaggggctagaattc

SEQ ID NO: 40 - DNA, 131 bp, Artificial Sequence
  Description of Artificial Sequence Synthetic polynucleotide
ctcgagaaggtatattgctgttgacagtgagcgaaagagtcactgtctgaatgaatagtgaagccacagatgtattcattcagacagtgactcttctgcctactgcctcggacttcaaggggctagaattc

SEQ ID NO: 41 - DNA, 131 bp, Artificial Sequence
  Description of Artificial Sequence Synthetic polynucleotide
ctcgagaaggtatattgctgttgacagtgagcgaaacagcctcgatttttaagaatagtgaagccacagatgtattcttaaaaatcgaggctgttctgcctactgcctcggacttcaaggggctagaattc

SEQ ID NO: 42 - DNA, 131 bp, Artificial Sequence
  Description of Artificial Sequence Synthetic polynucleotide
ctcgagaaggtatattgctgttgacagtgagcgcggactggttatagatttataatagtgaagccacagatgtattataaatctataaccagtccatgcctactgcctcggacttcaaggggctagaattc

SEQ ID NO: 43 - DNA, 97 bp, Artificial Sequence
  Description of Artificial Sequence Synthetic oligonucleotide
tgctgttgacagtgagcgacaggacttcaacactatgttttagtgaagccacagatgtaaaacatagtgttgaagtcctggtgcctactgcctcgga

SEQ ID NO: 44 - DNA, 97 bp, Artificial Sequence
  Description of Artificial Sequence Synthetic oligonucleotide
tgctgttgacagtgagcgcaaggagaaagacaagaaggaatagtgaagccacagatgtattccttcttgtctttctccttttgcctactgcctcgga

SEQ ID NO: 45 - DNA, 97 bp, Artificial Sequence; identical to SEQ ID NO: 44
  Description of Artificial Sequence Synthetic oligonucleotide

SEQ ID NO: 46 - DNA, 97 bp, Artificial Sequence
  Description of Artificial Sequence Synthetic oligonucleotide
tgctgttgacagtgagcgacaaggtaattgcagtaatgaatagtgaagccacagatgtattcattactgcaattaccttggtgcctactgcctcgga

SEQ ID NO: 47 - DNA, 97 bp, Artificial Sequence
  Description of Artificial Sequence Synthetic oligonucleotide
tgctgttgacagtgagcgacagaaagagcacaacagagaatagtgaagccacacatgtattctctgttgtgctctttctggtgcctactgcctcgga

SEQ ID NO: 48 - DNA, 97 bp, Artificial Sequence
  Description of Artificial Sequence Synthetic oligonucleotide
tgctgttgacagtgagcgcccagatgttctttgccaataatagtgaagccacagatgtattattggcaaagaacatctggatgcctactgcctcgga

SEQ ID NO: 49 - DNA, 97 bp, Artificial Sequence; identical to SEQ ID NO: 48
  Description of Artificial Sequence Synthetic oligonucleotide

SEQ ID NO: 50 - DNA, 97 bp, Artificial Sequence
  Description of Artificial Sequence Synthetic oligonucleotide
tgctgttgacagtgagcgagcagcgtcacacagaagcatatagtgaagccacagatgtatatgcttctgtgtgacgctgcgtgcctactgcctcgga

SEQ ID NO: 51 - DNA, 97 bp, Artificial Sequence
  Description of Artificial Sequence Synthetic oligonucleotide
tgctgttgacagtgagcgaccgcctcttctttgagttctatagtgaagccacagatgtatagaactcaaagaagaggcggctgcctactgcctcgga

SEQ ID NO: 52 - DNA, 97 bp, Artificial Sequence
  Description of Artificial Sequence Synthetic oligonucleotide
tgctgttgacagtgagcgcggcatccactgtgaatgataatagtgaagccacagatgtattatcattcacagtggatgccatgcctactgcctcgga

SEQ ID NO: 53 - DNA, 97 bp, Artificial Sequence; identical to SEQ ID NO: 52
  Description of Artificial Sequence Synthetic oligonucleotide

SEQ ID NO: 54 - DNA, 97 bp, Artificial Sequence
  Description of Artificial Sequence Synthetic oligonucleotide
tgctgttgacagtgagcgactggtgctaattcctctcatatagtgaagccacagatgtatatgagaggaattagcaccagctgcctactgcctcgga

SEQ ID NO: 55 - DNA, 97 bp, Artificial Sequence
  Description of Artificial Sequence Synthetic oligonucleotide
tgctgttgacagtgagcgctagctgttgaagaaatattaatagtgaagccacagatgtattaatatttcttcaacagctattgcctactgcctcgga

SEQ ID NO: 56 - DNA, 97 bp, Artificial Sequence
  Description of Artificial Sequence Synthetic oligonucleotide
tgctgttgacagtgagcgcaaggtgaagttgtctcgtttatagtgaagccacagatgtataaacgagacaacttcaccttatgcctactgcctcgga

SEQ ID NO: 57 - DNA, 97 bp, Artificial Sequence
  Description of Artificial Sequence Synthetic oligonucleotide
tgctgttgacagtgagcgcccgcctgaagtctctgattaatagtgaagccacagatgtattaatcagagacttcaggcggttgcctactgcctcgga

SEQ ID NO: 58 - DNA, 97 bp, Artificial Sequence
  Description of Artificial Sequence Synthetic oligonucleotide
tgctgttgacagtgagcgactggacgaactgataatgctatagtgaagccacagatgtatagcattatcagttcgtccaggtgcctactgcctcgga

SEQ ID NO: 59 - DNA, 97 bp, Artificial Sequence
  Description of Artificial Sequence Synthetic oligonucleotide
tgctgttgacagtgagcgcaagaaacttggtgttaggtaatagtgaagccacagatgtattacctaacaccaagtttcttttgcctactgcctcgga

SEQ ID NO: 60 - DNA, 97 bp, Artificial Sequence
  Description of Artificial Sequence Synthetic oligonucleotide
tgctgttgacagtgagcgcacgacgagaacagttgaaacatagtgaagccacagatgtatgtttcaactgttctcgtcgtttgcctactgcctcgga

SEQ ID NO: 61 - DNA, 97 bp, Artificial Sequence
  Description of Artificial Sequence Synthetic oligonucleotide
tgctgttgacagtgagcgaccaccatcttgtgtacatctttagtgaagccacagatgtaaagatgtacacaagatggtggctgcctactgcctcgga

SEQ ID NO: 62 - RNA, 5 nt, Artificial Sequence
  Description of Artificial Sequence Synthetic oligonucleotide
dcnnc

SEQ ID NO: 63 - RNA, 7 nt, Artificial Sequence
  Description of Artificial Sequence Synthetic oligonucleotide
acuucaa

SEQ ID NO: 64 - RNA, 7 nt, Artificial Sequence
  Description of Artificial Sequence Synthetic oligonucleotide
gcuucga

SEQ ID NO: 65 - RNA, 7 nt, Artificial Sequence
  Description of Artificial Sequence Synthetic oligonucleotide
ucuucug

SEQ ID NO: 66 - RNA, 6 nt, Artificial Sequence
  Description of Artificial Sequence Synthetic oligonucleotide
gaauuc

SEQ ID NO: 67 - RNA, 6 nt, Artificial Sequence
  Description of Artificial Sequence Synthetic oligonucleotide
gacuuc

SEQ ID NO: 68 - DNA, 122 bp, Homo sapiens
ctaaagaaggtatattgctgttgacagtgagcgactgtaaacatcctcgactggaagctgtgaagccacagatgggctttcagtcggatgtttgcagctgcctactgcctcggacttcaagg

SEQ ID NO: 69 - DNA, 120 bp, Artificial Sequence
  Description of Artificial Sequence Synthetic polynucleotide
ctcgagaaggtatattgctgttgacagtgagcgnnnnnnnnnnnnnnnnnnnnnntagtgaagccacagatgtannnnnnnnnnnnnnnnnnnnnntgcctactgcctcggaattcaagg

SEQ ID NO: 70 - RNA, 151 nt, Artificial Sequence
  Description of Artificial Sequence Synthetic polynucleotide
gguuaacccaacagaaggcucgagaagguauauugcuguugacagugagcgnnnnnnnnnnnnnnnnnnnnnnuagugaagccacagauguannnnnnnnnnnnnnnnnnnnnnugccuacugccucggaauucaaggggcuacuuuagga

SEQ ID NO: 71 - RNA, 153 nt, Homo sapiens
gguuaacccaacagaaggcuaaagaagguauauugcuguugacagugagcgacuguaaacauccucgacuggaagcugugaagccacagaugggcuuucagucggauguuugcagcugccuacugccucggacuucaaggggcuacuuuagga

SEQ ID NO: 72 - RNA, 157 nt, Artificial Sequence
  Description of Artificial Sequence Synthetic polynucleotide
ucuucagguuaacccaacagaaggcucgagaagguauauugcuguugacagugagcgnnnnnnnnnnnnnnnnnnnnnnuagugaagccacagauguannnnnnnnnnnnnnnnnnnnnnugccuacugccucggaauucaaggggcuacuuuagga

SEQ ID NO: 73 - DNA, 157 bp, Artificial Sequence
  Description of Artificial Sequence Synthetic oligonucleotide
ucgacttcuuaacccaacagaaggcucgagaagguauauugcuguugacagugagcgnnnnnnnnnnnnnnnnnnnnnnuagugaagccacagauguannnnnnnnnnnnnnnnnnnnnnugccuacugccucggacuucaaggggcuagaauucga

SEQ ID NO: 74 - RNA, 170 nt, Homo sapiens
uuacuucuucagguuaacccaacagaaggcuaaagaagguauauugcuguugacagugagcgacuguaaacauccucgacuggaagcugugaagccacagaugggcuuucagucggauguuugcagcugccuacugccucggacuucaaggggcuacuuuaggagcaauu

SEQ ID NO: 75 - RNA, 168 nt, Artificial Sequence
  Description of Artificial Sequence Synthetic polynucleotide
uuacuucuucagguuaacccaacagaaggcucgacaagguauauugcuguugacagugagcgcuggagaaguuuacuacauaaauacugaagccagagauguauuuauguaguaaacuucuccauugccuacugccucggaauucaaggggcuacuuuaggagcaauu

SEQ ID NO: 76 - RNA, 168 nt, Artificial Sequence
  Description of Artificial Sequence Synthetic polynucleotide
uuacuucgacuucuuaacccaacagaaggcucgacaagguauauugcuguugacagugagcgcuggagaaguuuacuacauaaauagugaagccacagauguauuuauguaguaaacuucuccauugccuacugccucggacuucaaggggcuagaauucgagcaauu

SEQ ID NO: 77 - RNA, 150 nt, Artificial Sequence
  Description of Artificial Sequence Synthetic polynucleotide
cuuaggagccuguuccucucacgcccucaccuggcugagccgcaguaguucuucaguggcaagcuuuauguccugacccagcuaaagcugccaguugaagaacuguugcccucugccccuggcuucgaggaggaggaggagcugcuuucc

SEQ ID NO: 78 - RNA, 10 nt, Artificial Sequence
  Description of Artificial Sequence Synthetic oligonucleotide
ccucaccugg

SEQ ID NO: 79 - RNA, 15 nt, Artificial Sequence
  Description of Artificial Sequence Synthetic oligonucleotide
ccccuggcuucgagg

SEQ ID NO: 80 - RNA, 239 nt, Artificial Sequence
  Description of Artificial Sequence Synthetic polynucleotide
uaaaaccuuggaguaaaguggcagcacauaaugguuuguggauuuugaaaaggugcaggccauauugugcugccucaaaaauacaaggaucugaucuucugaagaaaauauauggcuuuuuauucauagcucuuaugauagcaaugucagcagugccuuagcagcacguaaauauuggcguuaagauucuaaaauuaucuccaguauuaacugugcugcugaaguaagguugaccauac

SEQ ID NO: 81 - RNA, 12 nt, Artificial Sequence
  Description of Artificial Sequence Synthetic oligonucleotide
ugaucuucugaa

SEQ ID NO: 82 - RNA, 11 nt, Artificial Sequence
  Description of Artificial Sequence Synthetic oligonucleotide
uuuuauucaua

SEQ ID NO: 83 - DNA, 157 bp, Homo sapiens
cttcaggttaacccaacagaaggctaaagaaggtatattgctgttgacagtgagcgactgtaaacatcctcgactggaagctgtgaagccacagatgggctttcagtcggatgtttgcagctgcctactgcctcggacttcaaggggctactttagg

SEQ ID NO: 84 - DNA, 155 bp, Artificial Sequence
  Description of Artificial Sequence Synthetic polynucleotide
cttcaggttaacccaacagaaggctcgagaaggtatattgctgttgacagtgagcgcaggaattataatgcttatctatagtgaagccacagatgtatagataagcattataattcctatgcctactgcctcggaattcaaggggctactttagg

SEQ ID NO: 85 - DNA, 155 bp, Artificial Sequence
  Description of Artificial Sequence Synthetic polynucleotide
cttcaggttaacccaacagaaggctcgagaaggtatattgctgttgacagtgagcgaccagctaaaggtgaagatatatagtgaagccacagatgtatatatcttcacctttagctggctgcctactgcctcggaattcaaggggctactttagg

SEQ ID NO: 86 - DNA, 155 bp, Artificial Sequence
  Description of Artificial Sequence Synthetic polynucleotide
cttcaggttaacccaacagaaggctcgagaaggtatattgctgttgacagtgagcgacagctaaaggtgaagatatattagtgaagccacagatgtaatatatcttcacctttagctggtgcctactgcctcggaattcaaggggctactttagg

SEQ ID NO: 87 - DNA, 155 bp, Artificial Sequence
  Description of Artificial Sequence Synthetic polynucleotide
cttcaggttaacccaacagaaggctcgagaaggtatattgctgttgacagtgagcgacagctaaaggtgaagatatatctgtgaagccacagatgggatatatcttcacctttagctggtgcctactgcctcggaattcaaggggctactttagg

SEQ ID NO: 88 - DNA, 153 bp, Artificial Sequence
  Description of Artificial Sequence Synthetic polynucleotide
cttcaggttaacccaacagaaggctcgagaaggtatattgctgttgacagtgagcgacagctaaatgaagatatattagtgaagccacagatgtaatatatcttcacctttagctggtgcctactgcctcggaattcaaggggctactttagg

SEQ ID NO: 89 - DNA, 153 bp, Artificial Sequence
  Description of Artificial Sequence Synthetic polynucleotide
cttcaggttaacccaacagaaggctcgagaaggtatattgctgttgacagtgagcgacagctaaaggaagatatattagtgaagccacagatgtaatatatcttcacctttagctggtgcctactgcctcggaattcaaggggctactttagg

SEQ ID NO: 90 - DNA, 153 bp, Artificial Sequence
  Description of Artificial Sequence Synthetic polynucleotide
cttcaggttaacccaacagaaggctcgagaaggtatattgctgttgacagtgagcgacagctaaatgaagatatatctgtgaagccacagatgggatatatcttcacctttagctggtgcctactgcctcggaattcaaggggctactttagg

SEQ ID NO: 91 - DNA, 153 bp, Artificial Sequence
  Description of Artificial Sequence Synthetic polynucleotide
cttcaggttaacccaacagaaggctcgagaaggtatattgctgttgacagtgagcgacagctaaaggaagatatatctgtgaagccacagatgggatatatcttcacctttagctggtgcctactgcctcggaattcaaggggctactttagg

SEQ ID NO: 92 - DNA, 159 bp, Artificial Sequence
  Description of Artificial Sequence Synthetic polynucleotide
cttcaggttaacccaacagaaggctcgagaaggtatattgctgttgacagtgagcgacatatatcttcacctttagctggctgtgaagccacagatgggccagctaaaggtgaagatatatgctgcctactgcctcggaattcaaggggctactttagg

SEQ ID NO: 93 - DNA, 155 bp, Artificial Sequence
  Description of Artificial Sequence Synthetic polynucleotide
cttcaggttaacccaacagaaggctcgagaaggtatattgctgttgacagtgagcgacatatatcttcacctttagctgggtgaagccacagatgccagctaaaggtgaagatatatgctgcctactgcctcggaattcaaggggctactttagg

SEQ ID NO: 94 - DNA, 157 bp, Artificial Sequence
  Description of Artificial Sequence Synthetic polynucleotide
cttcaggttaacccaacagaaggctcgagaaggtatattgctgttgacagtgagcgacatatatcttcacctttagctggctgtgaagccacagatgggccagctaaatgaagatatatgctgcctactgcctcggaattcaaggggctactttagg

SEQ ID NO: 95 - DNA, 153 bp, Artificial Sequence
  Description of Artificial Sequence Synthetic polynucleotide
cttcaggttaacccaacagaaggctcgagaaggtatattgctgttgacagtgagcgacatatatcttcacctttagctgggtgaagccacagatgccagctaaatgaagatatatgctgcctactgcctcggaattcaaggggctactttagg

SEQ ID NO: 96 - DNA, 153 bp, Artificial Sequence
  Description of Artificial Sequence Synthetic polynucleotide
cttcaggttaacccaacagaaggctcgagaaggtatattgctgttgacagtgagcgacatatatcttcacctttagctgggtgaagccacagatgccagctaaaggaagatatatgctgcctactgcctcggaattcaaggggctactttagg

SEQ ID NO: 97 - DNA, 155 bp, Artificial Sequence
  Description of Artificial Sequence Synthetic polynucleotide
cgacttcttaacccaacagaaggctcgagaaggtatattgctgttgacagtgagcgacagctaaaggtgaagatatattagtgaagccacagatgtaatatatcttcacctttagctggtgcctactgcctcggacttcaaggggctagaattcg

SEQ ID NO: 98 - DNA, 155 bp, Artificial Sequence
  Description of Artificial Sequence Synthetic polynucleotide
cgacttcttaacccaacagaaggctcgagaaggtatattgctgttgacagtgagcgacagctaaaggtgaagatatatctgtgaagccacagatgggatatatcttcacctttagctggtgcctactgcctcggacttcaaggggctagaattcg

SEQ ID NO: 99 - DNA, 157 bp, Artificial Sequence
  Description of Artificial Sequence Synthetic polynucleotide
cgacttcttaacccaacagaaggctcgagaaggtatattgctgttgacagtgagcgacatatatcttcacctttagctggctgtgaagccacagatgggccagctaaatgaagatatatgctgcctactgcctcggacttcaaggggctagaattcg

SEQ ID NO: 100 - DNA, 153 bp, Artificial Sequence
  Description of Artificial Sequence Synthetic polynucleotide
cgacttcttaacccaacagaaggctcgagaaggtatattgctgttgacagtgagcgacatatatcttcacctttagctgggtgaagccacagatgccagctaaaggaagatatatgctgcctactgcctcggacttcaaggggctagaattcg

(19) United States
(12) Patent Application Publication
(10) Pub. No.: US 2015/0018539 A1
(43) Pub. Date: Jan. 15, 2015

(54) MODIFIED MIRNA AS A SCAFFOLD FOR SHRNA

(71) Applicant: Mirimus, Inc., Cold Spring Harbor, NY (US)

(72) Inventor: Christof Fellmann, Huntington, NY (US)

(21) Appl. No.: 14/310,753

(22) Filed: Jun. 20, 2014

Related U.S. Application Data
(63) Continuation of application No. PCT/US2014/013090, filed on Jan. 26, 2014.
(60) Provisional application No. 61/757,104, filed on Jan. 26, 2013.

(51) Int. Cl. C12N 15/113

(52) U.S. Cl. CPC C12N 15/113; C12N 2310/141
USPC 536/24.5; 435/320.1

(57) ABSTRACT

What is described is a modified miRNA molecule for producing an artificial siRNA/mature small RNA molecule that inhibits the expression of a target transcript of a host cell, comprising a stem region modified to comprise a sequence encoding the artificial siRNA molecule, consisting of a guide and a passenger strand; a conserved region having specific sequences; and a nonconserved region modified to include a recognition site for a restriction enzyme while preserving the native secondary structure of the miRNA. The modified miRNA molecule produced with these elements substantially inhibits the expression of the target transcript when expressed from an endogenous or exogenous promoter in the host cell.

### CROSS-REFERENCE TO RELATED APPLICATIONS

This is a continuation application of International Patent Application No. PCT/US2014/013090 filed Jan. 26, 2014, which application claims the benefit of U.S. Provisional Patent Application No. 61/757,104, filed Jan. 26, 2013, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 3, 2014, is named 101756.000006_SL.txt and is 36,179 bytes in size.

### TECHNICAL FIELD

RNA interference, gene knockdown, regulation of gene expression, genetic engineering, RNA biology, small RNA biology, genetics, genetic manipulation.

Application fields: Molecular biology, cancer biology, study of disease biology, biomedical sciences, life sciences, therapeutic studies, drug target research, drug target development.

### BACKGROUND

RNA interference (RNAi) is an endogenous pathway that fine tunes gene expression (among others). This pathway provides a powerful tool to suppress the expression of a specific gene at will, for a specific period of time, reversibly. It can be used to uncover gene function and biochemical pathways and/or to test new therapeutic strategies. To implement RNAi as a tool, various triggers can be used, including siRNAs, stem-loop shRNAs, and miRNA-embedded shRNAs. MicroRNA-embedded shRNAs that use a miRNA backbone resemble most closely the endogenous triggers, and are least toxic and thus preferable. However, potent shRNA sequences must be identified among hundreds to thousands of possibilities; thus they are rare and hard to identify.

Conventional stem loop shRNA structures provide a simple si-based design and in many cases potent knockdown. However, these shRNA molecules are often toxic, lack a robust regulatable system, and are more prone to off-targets effects, e.g. due to imprecision in Dicer processing or saturation of Exportin-5 export.

Target transcript knockdown efficiency is a measure of potent, clearly controlled knockdown. Achieving knockdown efficiency depends on i) delivery and expression of the precursor of the RNAi trigger, e.g., a vector coding for a miRNA-embedded shRNA, ii) the processing efficiency of the precursor molecule (biogenesis, thus its ability to become a mature small RNA that can trigger an RNAi response), and iii) the propensity of the chosen shRNA sequence itself to elicit a strong target knockdown through mature RISC complexes. There is a need to generate microRNA (miRNA) backbones that lead to efficient/better processing of the synthetic miRNA precursor molecules, to increase the overall percentage of sequences that lead to potent target knockdown.

### SUMMARY

One aspect of the description is a modified miRNA molecule for producing an artificial siRNA (or shRNA) molecule that inhibits the expression of a target transcript, comprising
a stem region modified to comprise a sequence encoding the artificial siRNA molecule, consisting of a guide and a passenger strand;
a conserved region comprising a sequence consisting of 5′-DC*NNC-3′, wherein N consists of A, G, U, or C, D consists of A, G or U, and wherein the C* is located at position 16-20 of the 3′ arm;
   wherein the modified miRNA molecule substantially inhibits the target transcript when integrated into a host cell genome and expressed by the host cell. The conserved region preferably comprises the sequence 5′-ACUUCAA-3′,5′-GCUUCGA-3′, or 5′-UCUUCUG-3′.

One embodiment of the description is when the modified miRNA further comprises a region consisting of a recognition site for a restriction enzyme. Another embodiment is a modified miRNA wherein the native secondary structure is conserved. Another embodiment is when a modified miRNA molecule is derived from miR-30, preferably in which the region comprises the sequence 5′-CUUUAG-3′. In another embodiment, the recognition site consists of a sequences recognized by EcoRI or XhoI. When the restriction enzyme consists of EcoRI, the modified sequence may consist of 5′-GACUUC-3′ and the partially complementary sequence containing the EcoRI restriction sites consists of 5′-GAAUUC-3′. The modified miR-30 molecule may further comprise a modified loop region. A bulge in the guide strand may be absent from stem region of the modified miR-30 molecule.

In another embodiment, the miRNA molecule is derived from a miRNA selected from miR-30, miR-22, miR-15, miR-16, miR-103, and miR-107. In another embodiment the sequence of the conserved region is modified from the sequence 5′-ACUUCAA-3′ to a sequence selected from the group consisting of 5′-ACGUCAA-3′,5′-ACCUCAA-3′, and 5′-ACAUCAA-3′.

In another embodiment, the modified miRNA molecule may be derived from miR-22. In this connection, the conserved region may comprise the sequence 5′-GCUUCGA-3′.

In another embodiment, the modified miRNA molecule is derived from miR-15/16.

In another embodiment, the modified miRNA molecule consists of a precursor miRNA molecule, preferably a pri-miRNA molecule. The modified pri-miRNA molecule may be efficiently processed by Drosha into a pre-miRNA in the nucleus of a host cell expressing the modified miRNA. The pre-miRNA molecule may be subsequently processed by Dicer in the cytoplasm of the host cell.

In another aspect of the description, a nucleic acid construct encodes the modified miRNA. In one embodiment, the Pol II promoter may be a constitutive promoter, an inducible promoter, a ubiquitous promoter, a tissue-specific promoter, and/or a developmental stage-specific promoter. In another embodiment, the Pol II promoter may be a CMV-derived promoter. The nucleic acid construct may further comprise at least one selectable marker. The nucleic acid construct may further comprise a reporter transcript. The nucleic acid construct may further comprise a Pol III promoter upstream of the coding sequence for expressing the precursor molecule.

In another embodiment of the description, a cell may comprise the nucleic acid construct encoding the modified miRNA. In another embodiment the cell may be a mammalian cell, or another vertebrate cell (e.g. a chicken cell). The cell may be cultured in vitro (e.g. in a plastic dish), or be part of an organism, including a mammal or other vertebrate. In this respect, another embodiment of the description herein is in the design of a therapeutic for use in treating a disease, or in the design of a therapeutic.

Another embodiment of the description is a method for inhibiting the expression of a target transcript of interest in a cell-free system, or in a cell, comprising introducing a nucleic acid construct encoding the modified miRNA into the cell, wherein the siRNA molecule derived from the modified miRNA molecule is specific for the target transcript. In another embodiment, the method further comprises inhibiting at least one additional target transcript(s) of interest in the cell by introducing at least one additional nucleic acid construct encoding another modified miRNA molecule into the cell, wherein each of the siRNA molecules derived from the modified miRNA molecule is specific for the additional target transcripts, respectively.

Another embodiment of the description is a method for treating a transcript-mediated disease, comprising introducing into an individual having the disease a nucleic acid construct encoding the modified miRNA molecule, where the siRNA derived from the modified miRNA molecule is specific for the transcript mediating the disease, or specific for an auxiliary transcript necessary for disease progression or maintenance.

Another embodiment of the description is a method of validating a transcript as a potential target for treating a disease, comprising: introducing a nucleic acid construct encoding the modified miRNA into a cell associated with the disease, wherein the siRNA molecule derived from the modified miRNA is specific for the transcript; and assessing the effect of inhibiting the expression of the transcript or gene surrogate on one or more disease-associated phenotypes; wherein a positive effect on at least one disease-associated phenotype is indicative that the transcript is a potential target for treating the disease.

Another embodiment of the description is a method for producing a modified miRNA molecule for inhibiting the expression of a target transcript by preparing a nucleic acid construct encoding the modified miRNA comprising the steps of
modifying the stem sequence of the miRNA molecule to substitute a nucleic acid sequence encoding a siRNA molecule;
substituting a conserved region comprising a sequence consisting of 5′-DC*NNC-3′, wherein N consists of A, G, U, or C, D consists of A, G or U, and wherein the C* is located at position 16-20 of the 3′ arm;
   wherein the modified miRNA molecule substantially inhibits the target transcript when the nucleic acid construct is integrated into a host cell genome. The conserved region preferably comprises the sequence 5′-ACUUCAA-3′,5′-GCUUCGA-3′, or 5′-UCUUCUG-3′.

An embodiment comprises modifying a region to include a recognition site for a restriction enzyme. Another embodiment is to preserve the miRNA secondary structure. Another embodiment is the method wherein a miRNA molecule is derived from miR-30 and the region comprises the sequence 5′-CUUUAG-3′. Further, the restriction enzyme preferably consists of EcoRI or XhoI, more preferably wherein the restriction enzyme consists of EcoRI, the modified sequence consists of GACUUC and the partially complementary sequence containing the EcoRI site consists of GAATTC. Alternatively, the miRNA molecule may be derived from miR-22 and the conserved region comprises the sequence 5′-GCUUCGA-3′ or may be derived from miR-15/16 and the conserved region comprises the sequence 5′-UCUUCUG3′.

### BRIEF DESCRIPTION OF THE DRAWINGS

FIG. 1 shows a comparison of core sequence of endogenous miR-30 (human MIR30A, above) (SEQ ID NO: 68) and synthetic miR-30 (below) (SEQ ID NO: 69). The regions of endogenous miR-30 in red in the upper sequence show completely conserved nucleotides. The regions in synthetic miR-30 that are altered in the conserved region are shown in red in the lower sequence. A synthetic sequence element (EcoRI site) in a 3′ conserved region, highlighted in blue in the lower, synthetic sequence, contains mutations in one highly conserved nucleotide.

FIG. 2A shows a representation of miR-30 (SEQ ID NO: 70) vs. miR-30A (SEQ ID NO: 71)

FIG. 2B shows a respresentation of miR-30 (SEQ ID NO: 72) vs. miR-E (SEQ ID NO: 73)

FIG. 2C shows a representation of the core structures of endogenous human miR-30A (SEQ ID NO: 74), synthetic miR-30 (SEQ ID NO: 75), and miR-E (SEQ ID NO: 76) (arrow, SNP (C>A); lower circle, relocated EcoRI site; middle circle, bulge in stem loop; upper circle, loop variation; miR-30 and miR-E are shown with sh.Yap1.891E)

FIG. 2D shows a representation of miR-22 (SEQ ID NOS 77-79, respectively, in order of appearance)

FIG. 2E shows a representation of miR-15/16 (SEQ ID NOS 80-82, respectively, in order of appearance).

FIG. 3 shows the results of modifying the conserved flank region of miR-30. Single point mutations were generated always exchanging one nucleotide. All constructs had the repositioned EcoRI site and 5′ arm adaptations. All were tested in NIH3T3 cells at single copy. Nucleotide substitutions are shown below the arrows. The conserved nucleotide (C) that was changed in the synthetic miR-30 is (indicated with an asterisk).

FIGS. 4A & B show the results.

FIG. 4A shows the sensor shRNA reporter assay system.

FIG. 4B shows results of modifying the stem loop region of miR-30. The miR-30 molecule without and with bulge was compared to miR-155 (BLOCK-iT, Invitrogen), which has a guide bulge. Two validated Luc.shmiR molecules were tested, shmir Luc.1309, originally validated in miR-30; and shmir Luc.201, a control shRNA from Invitrogen. All were cloned in pLMN and infected at idential single-copy conditions. The results of testing expression at day 1 (left panel) and day 6 (right panel) are shown.

FIG. 4C summarizes the results of FIG. 4B for shmir Luc.201 (left panel) and shmir.Luc.1309 (right panel). Percentage knockdown (vertical axis) relative to each shRNA⁻ is shown for 2 days (dark brown) and for 6 days (light brown).

FIG. 5A shows the sequence variants of the Pten.1524 shRNA (04-18) along with the Ren.713 (02) and Pten.1523 (03) control shRNAs. Human MIR30A (01) is shown for reference. FIG. 5A discloses SEQ ID NOS 83-100, respectively, in order of appearance.

| Endogenous sequences: | |
|---|---|
| 01) | Human MIR30A |
| Experimental sequences with single features: | |
| 02) | Ren.713 (standard miR-30 design) |
| 03) | Pten.1523 (standard miR-30 design) |
| 04) | Pten.1524 (standard miR-30 design) |
| 05) | Pten.1524 L (endogenous loop) |
| 06) | Pten.1524 B1 (bulge 1, contains a 2 nt bulge in the guide |
| | strand) |
| 07) | Pten.1524 B2 (bulge 2, contains a 2 nt bulge in the guide |
| | strand, positioned closer to the loop) |
| 08) | Pten.1524 L + B1 (endogenous loop and bulge 1) |
| 09) | Pten.1524 L + B2 (endogenous loop and bulge 2) |
| 10) | Pten.1524 R1 (reverse 1, swapped guide/passenger position, |
| | with a longer overall stem) |
| 11) | Pten.1524 R2 (reverse 2, swapped guide/passenger position) |
| 12) | Pten.1524 R1B1 (reverse 1 and bulge 1) |
| 13) | Pten.1524 R2B1 (reverse 2 and bulge 1) |
| 14) | Pten.1524 R2B2 (reverse 2 and bulge 2) |
| 15) | Pten.1524 E (endogenous 3′ flank at EcoRI site, |
| | repositioned EcoRI site, adapted 5′ complement region) |
| Experimental sequences with combined features, uniting single | |
| attributes that strongly enhance knockdown potency: | |
| 16) | Pten.1524 E + L (miR-E and endogenous loop) |
| 17) | Pten.1524 E + R1B1 (miR-E and reverse 1 with bulge 1) |
| 18) | Pten.1524 E + R2B2 (miR-E and reverse 2 with bulge 2) |

Structural microRNA regions are labeled and highlighted by shaded boxes. Guide strands are shown in green, and alterations highlighted in red. The XhoI/EcoRI restriction sites are underlined; the repositioned (“new”) EcoRI site and 5′ complement region are marked.

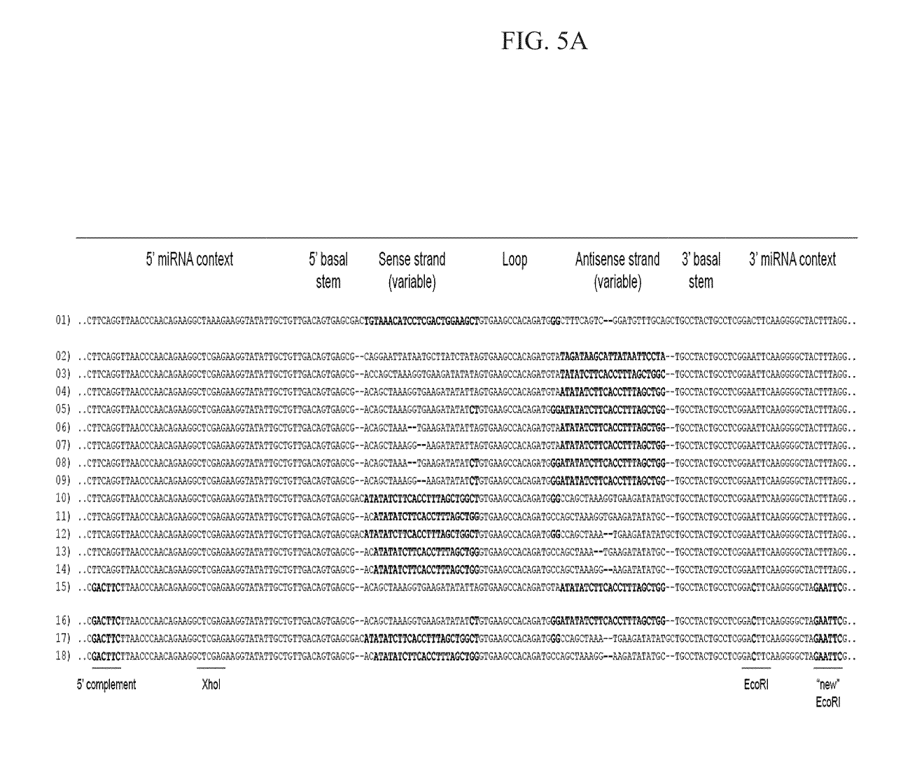

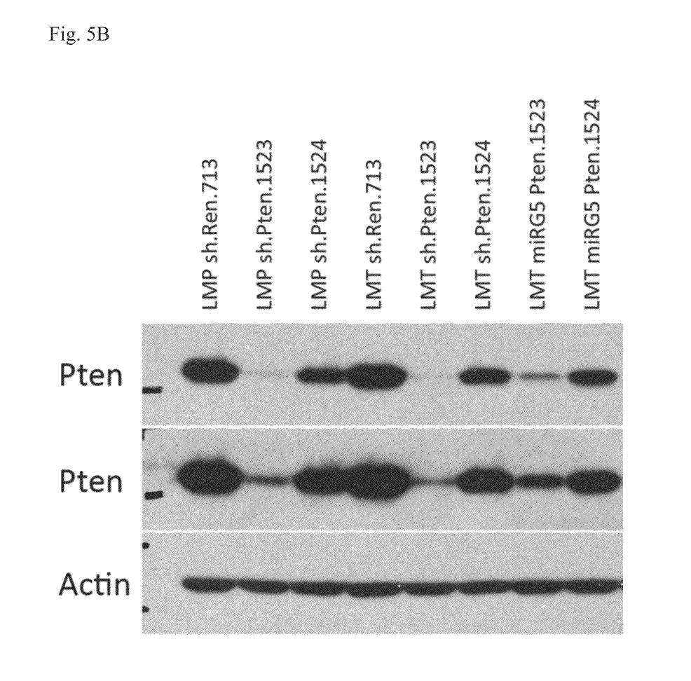

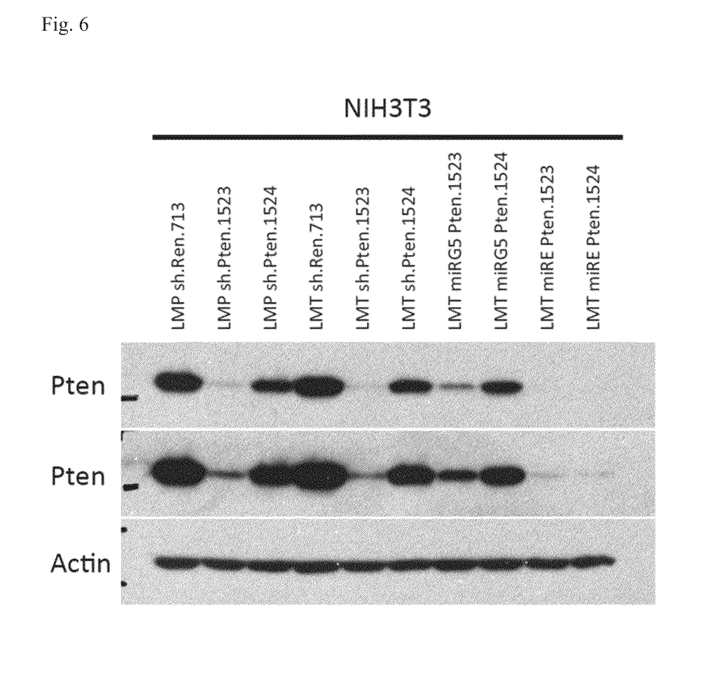

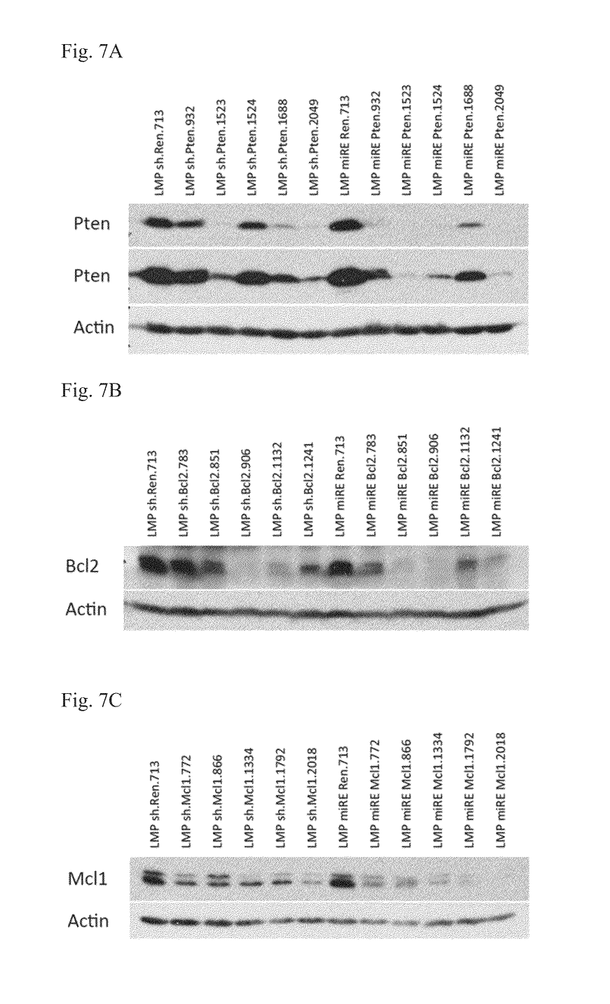

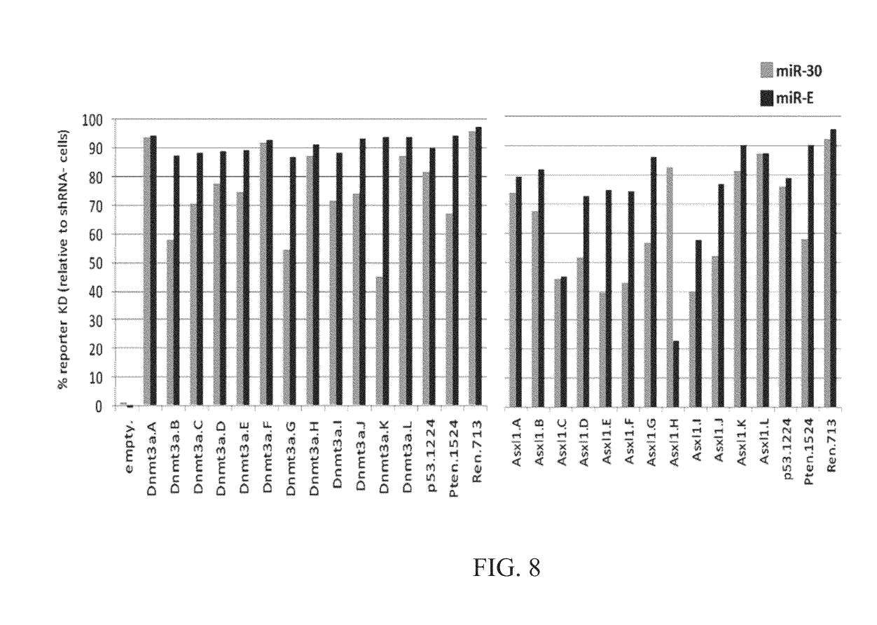

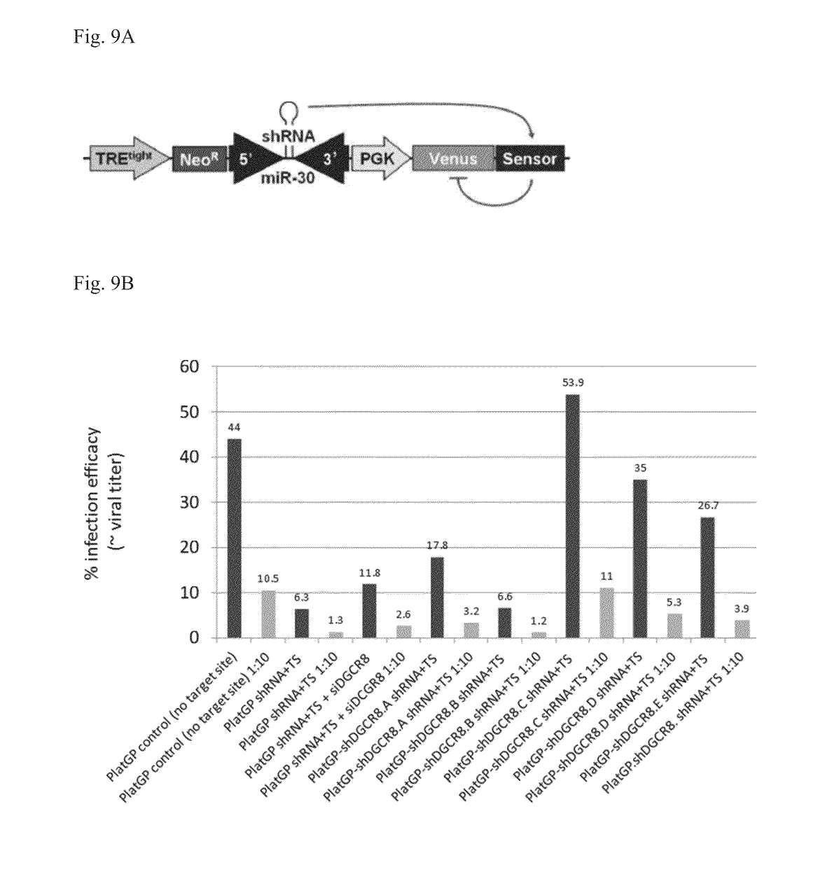

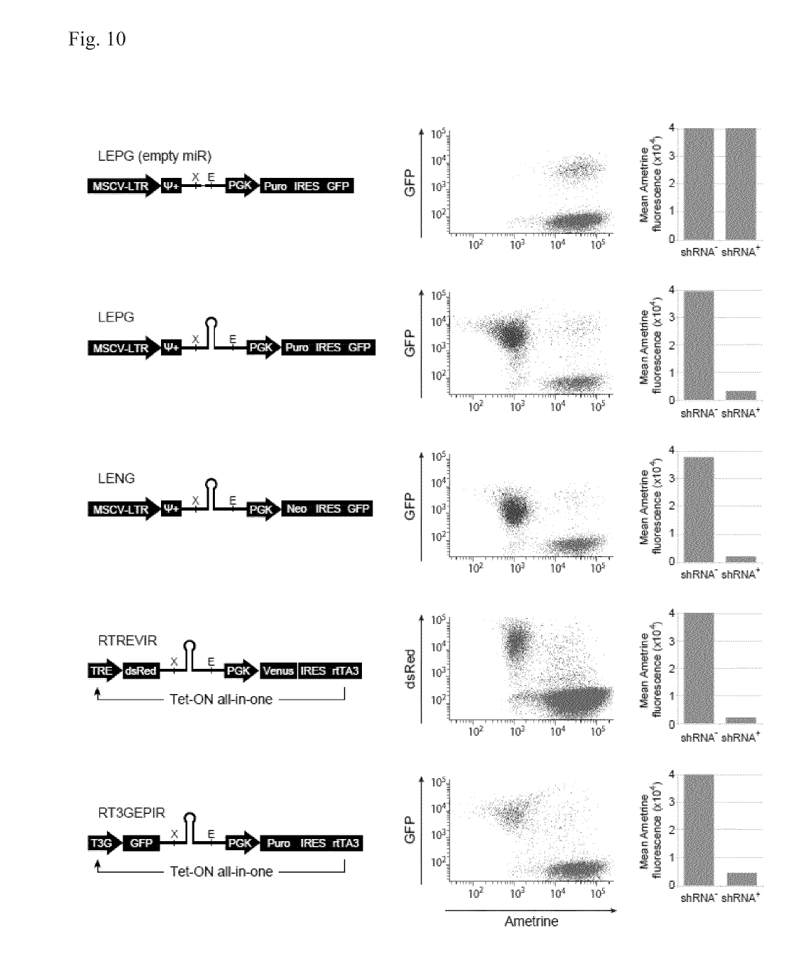

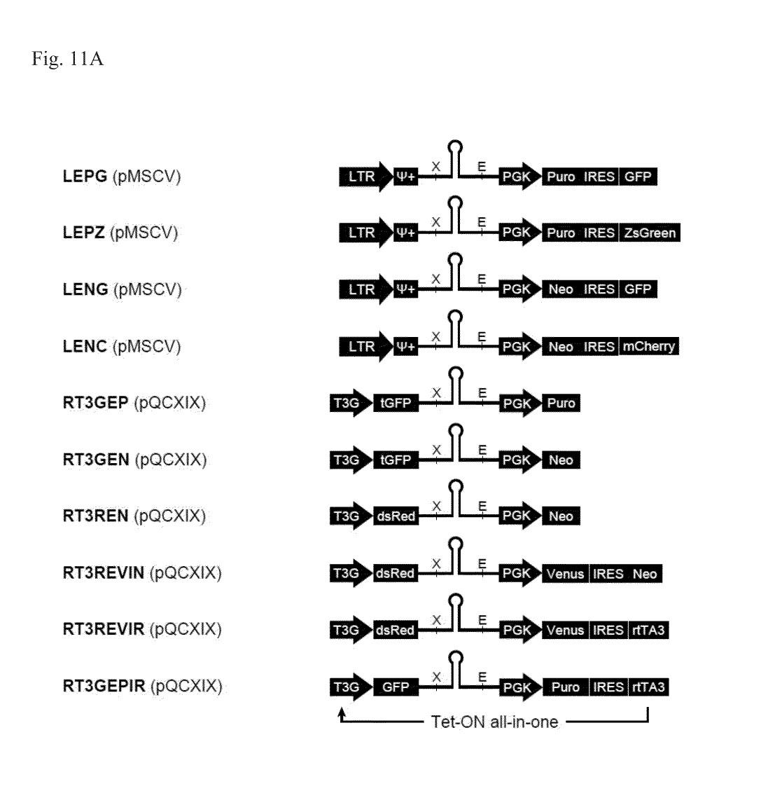

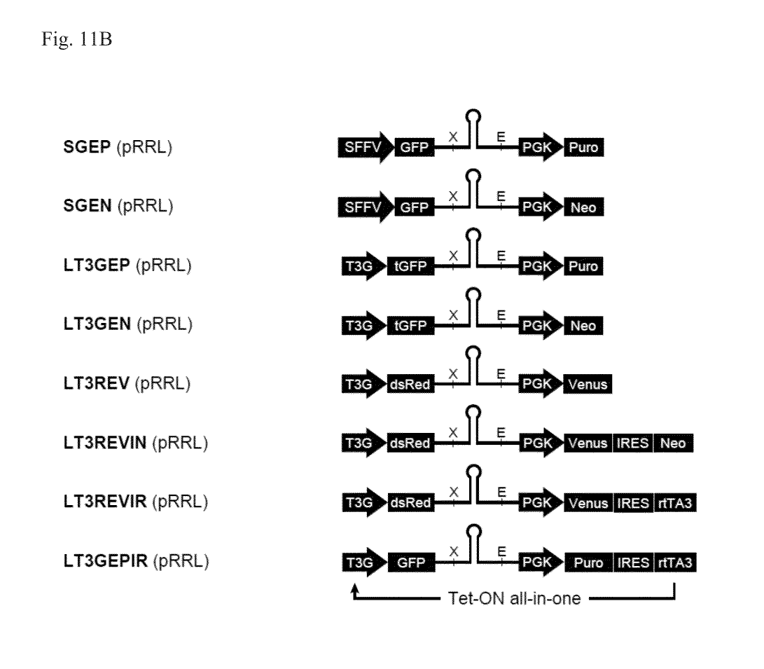

FIG. 5B shows the results of reversing the guide and passenger strands, and compares the knockdown for various miRNA sequences of FIG. 5A.

FIG. 6 shows the results of using miR-E as backbone to express shRNAs. miR-E expression of shRNAs targeting Pten (miRE Pten.1523 and miRE Pten.1524) is compared to the same shRNA sequences as synthetic miR-30 (sh.Pten.1523 and sh.Pten.1524) and miR-G5 (miRGS Pten.1523 and miRGS Pten.1524) constructs.

FIG. 7A shows the results of using miR-E to express a variety of shRNAs targeting different transcripts. The results of expressing various Pten shRNA are shown.

FIG. 7B shows the results of using miR-E to express a variety of shRNAs targeting different transcripts. The results of expressing various shRNA for knockdown of the Bc12 (B-cell CLL/lymphoma 2) gene are shown.

FIG. 7C shows the results of using miR-E to express a variety of shRNAs targeting different transcripts. The results of expressing various shRNA for knockdown of the Mc11 (Myeloid cell leukemia sequence 1) gene are shown.

FIG. 8 shows the results of testing the knockdown of a panel of shRNAs integrated at single copy. MiR-30 (grey) is compared to miR-E (black) for Dnmt3a shRNAs (left panel) and Asxll shRNAs (right panel). The percentage knockdown compared to shRNA negative cells is shown.

FIG. 9A shows the Sensor vector with the target site (labeled as “Sensor”).

FIG. 9B shows the rescue of viral packaging efficiency (viral titers) for Sensor vectors coding for a miRNA-embedded shRNA+it's target site, upon suppression of the RNAi pathway in a packaging cell line using stem-loop shRNA targeting components of the Drosha complex (specifically DGCR8). What is shown is the percentage infected cells, proportional to viral titer, resulting from packaging under a variety of conditions in PlatGP packaging cells. Suppression of the RNAi pathway using the stem loop shRNA shDGCR8.0 fully rescued packaging efficiency of Sensor vectors containing a target sites, as compared to Sensor vectors without target site.

FIG. 10 shows generation and validation of retro- and lentiviral vectors featuring miR-E. The results shows reporter-based validation of the miR-E shRNAs expression vectors shown in the left column. Reporter cells (e.g. NIH3T3s) were infected with a reporter construct constitutively expressing a transcript coding for Ametrine and harboring multiple target sites of established control shRNAs. Sorted Ametrine-positive cells were then infected with one of several retroviral expression vectors to be tested, expressing another fluorescent protein coupled to miR-E. After 6 days of culture (in presence of doxycycline for Tet-regulated vectors), levels of the Ametrine-reporter were quantified by flow cytometry in both shRNA⁺ and shRNA⁻ cells. The left column shows a schematic representation of tested retroviruses. The results of testing each vector are shown in the middle and right columns. While the vectors LEPG and LENG constitutively express the contained shRNA (Ren.713), the vectors RTREVIR and RT3GEPIR introduce rtTA3, allowing for doxycycline-regulated expression of the contained shRNA (Ren.713). Flow cytometry blots in the middle column show Ametrine levels at day 6 post-infection (constitutive vectors) or after 6 days of doxycycline treatment (inducible vectors). Bar graphs in the right column show the corresponding quantification of Ametrine levels.

FIG. 11A shows schematic maps of validated constitutive (pMSCV) and Tet-regulated (pQCXIX) retroviral miR-E expression vectors, featuring different drug selection and fluorescent markers.

FIG. 11B shows schematic maps of validated constitutive and Tet-regulated lentiviral miR-E expression vectors, featuring different drug selection and fluorescent markers.

### DETAILED DESCRIPTION OF ILLUSTRATIVE EMBODIMENTS

What is described herein is engineering of a new variant of an endogenous miRNA backbone, with drastically increased general knockdown efficiency. The increased knockdown efficiency results from the discovery of a conserved structure/sequence element in the 3′ flank of the miR-30 backbone, a sequence that had previously been unknown and mutated. What is further described is a means of engineering of a cloning site (e.g., EcoRI) in a non-conserved part of the backbone, and in a way that conserves the general secondary structure.

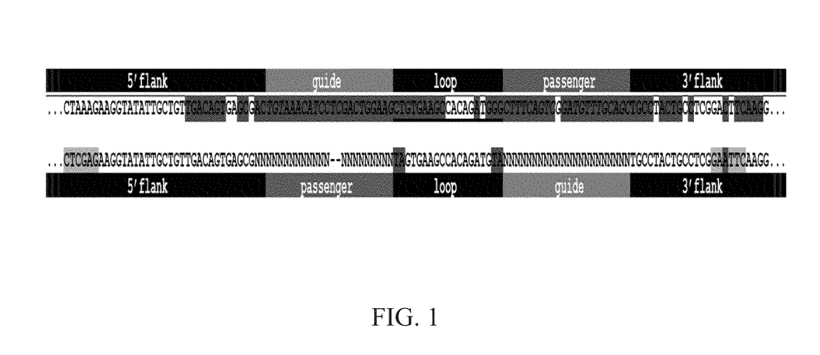

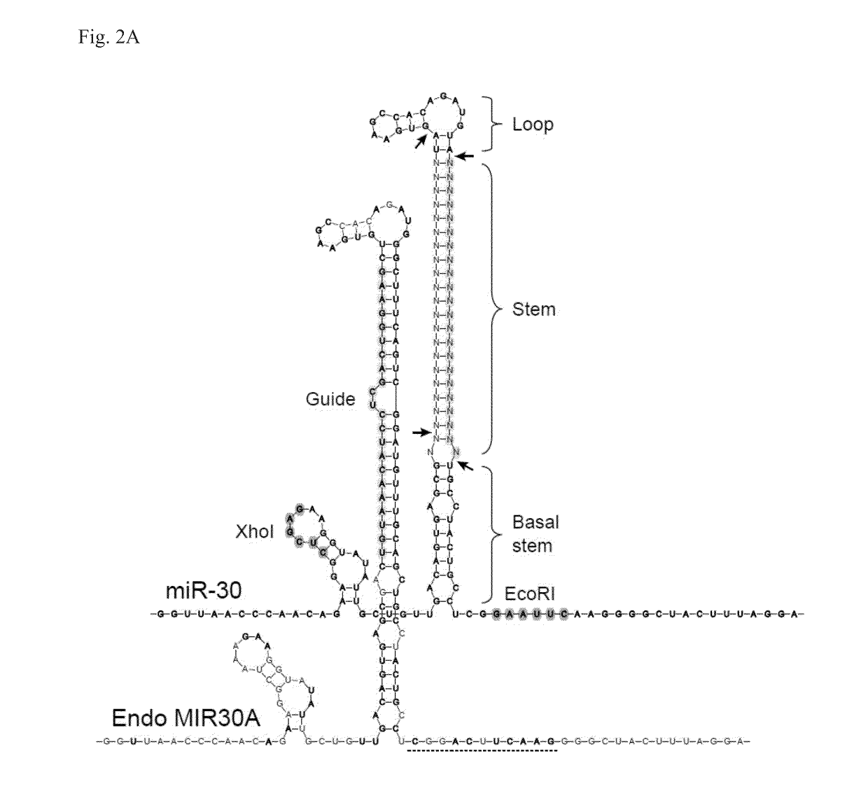

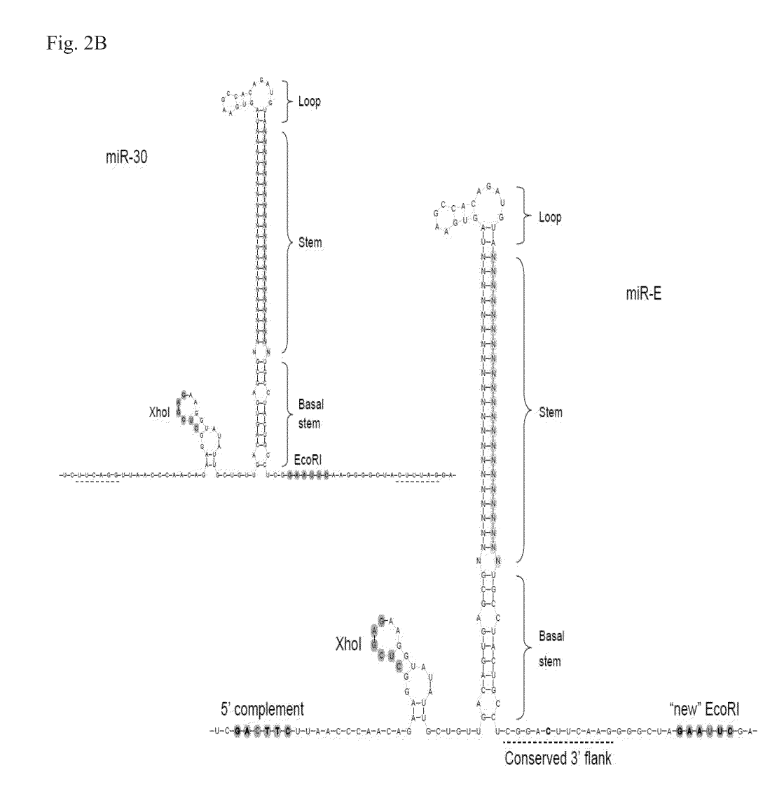

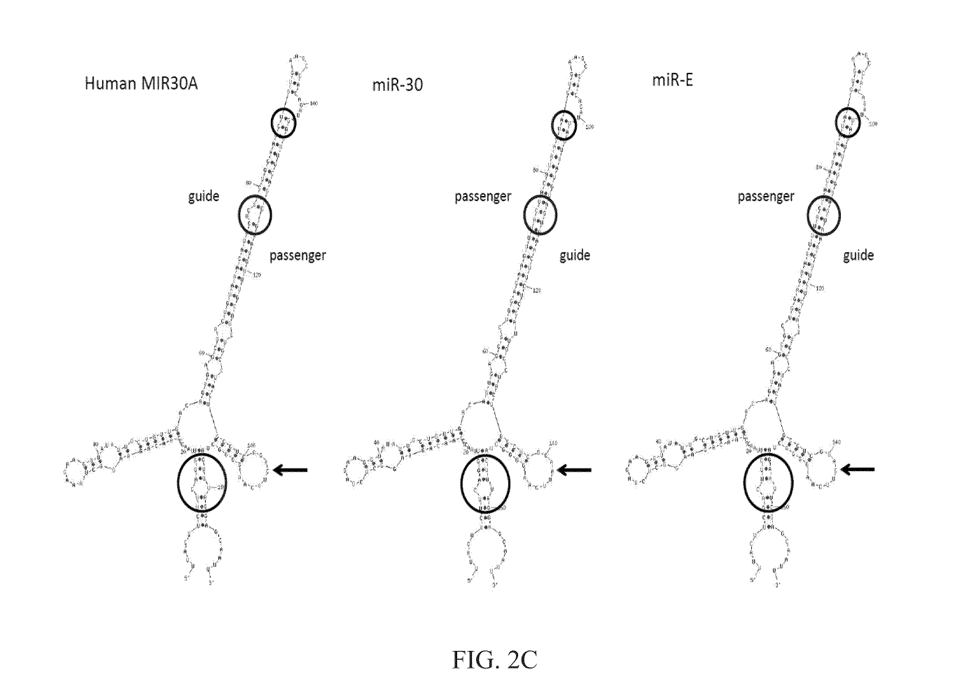

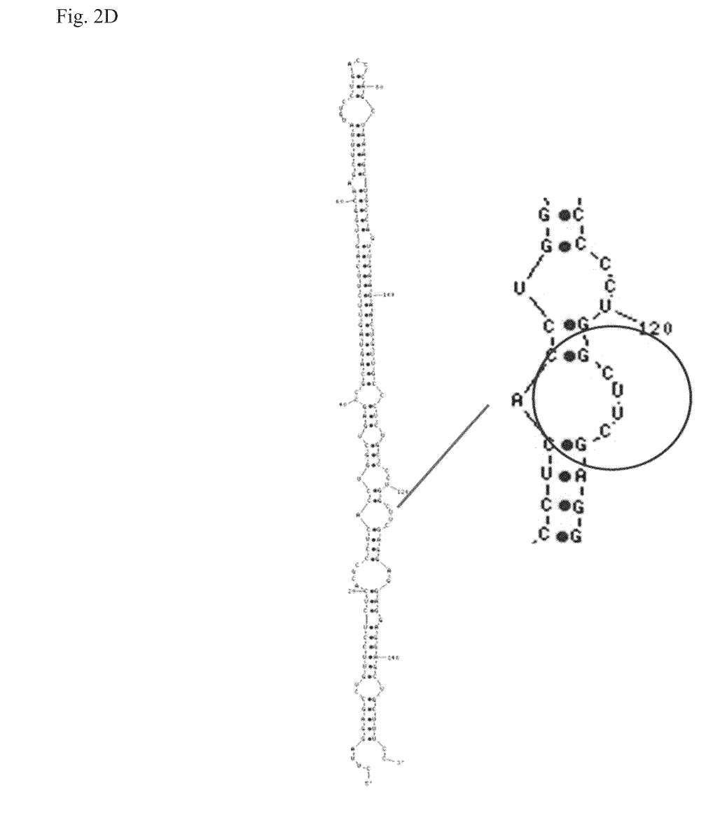

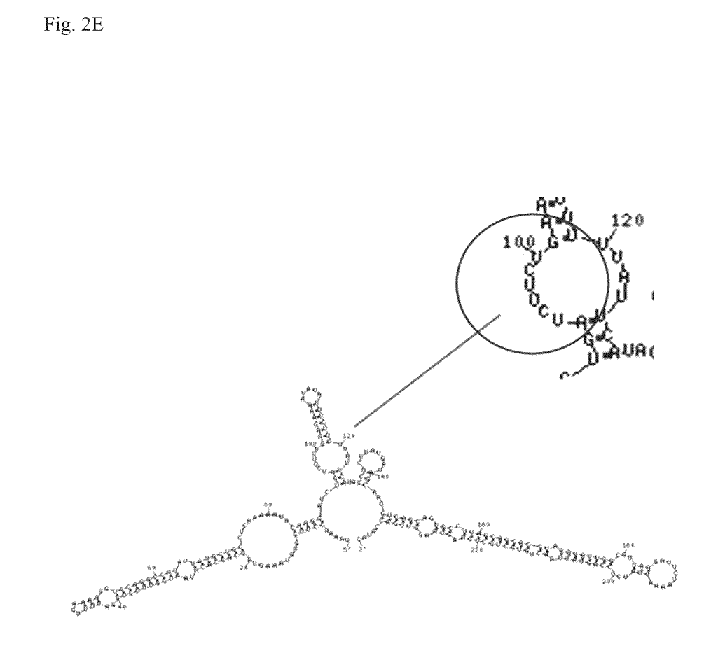

What is described is engineering of a new backbone of miRNA molecules that leads to better target knockdown through generally improved primary miRNA processing. This involved discovery of conserved sequence/structure elements required for efficient pri-miRNA processing. Implementation of conserved structures in an engineered backbone for efficient processing of synthetic RNAi triggers, leading to highly improved target knockdown. Specifically, what is described is an evolutionarily conserved region of miR-30 that is altered in synthetic miR-30 currently used as a scaffold for producing selected shRNA. This highly conserved region lies in the 3′ flank to the stem loop (FIG. 1 and FIG. 2A). A matching conserved region in miR-22 and mir-15/16 is also described herein (FIG. 2B and FIG. 2C, respectively).

What is further described is discovery and implementation of sequence/structure elements (as part of a miRNA backbone) that enhance primary miRNA processing. What is described herein is a combination of these conserved elements with strategies to insert cloning sites that do not affect knockdown efficiency, and assist easy and fast generation of synthetic shRNA vectors and libraries thereof.

What is described is a means of utilizing a miR-30-based system to overcome the limitations of simple stem loop systems. The modified miR-30 scaffold as a means of producing shRNA is less toxic in-vitro/in-vivo, is readily adaptable to a simple reportable Tet-system, and promises reduced off-target effects. The only downside that remains is that design of potent shRNAs is harder.

The issue is likely suboptimal pri-miRNA processing, because potent miR30-shRNAs are harder to find than potent stem-loop shRNAs, even Sensor-optimized shRNAs show huge variability in pri-miRNA processing, as evidenced in the Sensor deep-seq dataset and (indirectly) by variations in packaging efficacy and expression levels of spacer reporters, and even when expressed from strong promoters (e.g. TRE), mature small RNAs of synthetic miR30-shRNAs are less abundant than many endogenous miR5.

A technical issue is whether the synthetic miR30 structure can be further optimized to improve primary miRNA processing. What is described herein is a means of improving miRNA processing of miR-30 and thereby increasing knockdown efficiency.

RNA interference (RNAi) is normally triggered by double stranded RNA (dsRNA) or endogenous microRNA precursors (pri-miRNAs/pre-miRNAs). Since its discovery, RNAi has emerged as a powerful genetic tool for suppressing gene expression in mammalian cells. Stable gene knockdown can be achieved by expression of synthetic short hairpin RNAs (shRNAs).

The current modified miRNA can be produced from one single stably integrated expression construct. The single expression construct may be stably transfected/infected into a target cell, or may be a germline transgene. Transgenic animals with the subject RNAi constructs, which may be regulated to express miRNA-embedded shRNA in an inducible, reversible, constitutive and/or tissue-specific manner, can be used to establish valuable animal models for certain disease, such as those associated with loss-of-function of certain target genes, or to assess target genes for their effect on disease or normal physiology upon knockdown. The ability to control both the timing (e.g., at certain developmental stages) and location (e.g., tissue-specific) of target gene knock-down, including the ability to reverse the course of induction/inactivation, renders the subject system a powerful tool to study gene function and disease progression. Such animal models or cells thereof may also be used for drug and drug target screening or validation.

In certain embodiments, Pol II promoters control the transcription of the subject miRNA/shRNA coding sequence. In general, any Pol II compatible promoters may be used for the instant description. In certain embodiments, various inducible Pol II promoters may be used to direct precursor miRNA/shRNA expression. Exemplary inducible Pol II promoters include the tightly regulatable Tet system (either TetOn or TetOFF), and a number of other inducible expression systems known in the art and/or described herein. The tet systems allows incremental and reversible induction of precursor miRNA/shRNA expression in vitro and in vivo, with no or minimal leakiness in precursor miRNA/shRNA expression. Such inducible system has advantages over the existing unidirectional Cre-lox strategies. Other systems of inducible expression may also be used with the instant constructs and methods.

In certain embodiments, expression of the subject miRNA/shRNA may be under the control of a tissue specific promoter, such as a promoter that is specific for a variety of tissues including liver, pancreas (exocrine or endocrine portions), spleen, esophagus, stomach, large or small intestine, colon, GI tract, heart, lung, kidney, thymus, parathyroid, pineal gland, pituitary gland, mammary gland, salivary gland, ovary, uterus, cervix (e.g., neck portion), prostate, testis, germ cell, ear, eye, brain, retina, cerebellum, cerebrum, PNS or CNS, placenta, adrenal cortex or medulla, skin, lymph node, muscle, fat, bone, cartilage, synovium, bone marrow, epithelial, endothelial, vascular, and nervous tissues. The tissue specific promoter may also be specific for certain disease tissues, such as cancers. Any tissue specific promoters may be used in the connection with the nucleic acid constructs described herein.

In certain embodiments, artificial miRNA constructs based on, for example, miR30 (microRNA 30), may be used to express precursor miRNA/shRNA from single/low copy stable integration in cells in vivo, or through germline transmission in transgenic animals. In certain embodiments, even a single copy of stably integrated precursor miRNA/shRNA construct results in effective knockdown of a target gene. In certain embodiments, the inducible Tet system, coupled with the low-copy integration feature of description, allows more flexible screening applications, such as in screening for potentially lethal shRNAs or synthetic lethal shRNAs.

In certain embodiments, the subject precursor miRNA cassette may be inserted within a gene encoded by the subject vector. For example, the subject precursor miRNA coding sequence may be inserted with an intron, or in the 5′- or 3′-UTR of a reporter gene such as GFP.

In certain embodiments, cultured cells, such as wild type mouse fibroblasts or primary cells can be switched from proliferative to senescent states simply through regulated knockdown of p53 using the subject constructs and methods.

The constructs and methods of the description are advantageous in several respects. In one respect, stable precursor miRNA/shRNA expression may be effected through retroviral or lentiviral delivery of the miRNA/shRNAs, which is shown to be effective at single copy per cell. This allows very effective stable gene expression regulation at extremely low copy number per cell (e.g. one per cell), thus vastly advantageous over systems requiring the introduction of a large copy number of constructs into the target cell by, for example, transient transfection. High copy numbers can be achieved also through other means, e.g. viral transduction. In some instances single-copy is required, as for example pooled shRNA screens that require single-copy for the deconvolution of screening results, or in transgenic animals that may require single-copy for site specific integration of the transgene (expressing the shRNA) at a given locus.

Compared to transfection where there are multiple copies (such as multiple episomal copies) of the shRNA construct, and the LTR is active, the instant system is preferable for stable expression of the shRNA. A system described herein allows for stable expression, but could also be used in a transfection case. Even when infected and stably integrated into a target cell, the LTR promoter might be used (as is the case for example in the LMP vector, where the LTR promoter drives expression of the shRNA cassette). Another useful feature of the modified miRNA described herein is that it is compatible with an established miR30 miRNA/shRNA library, which contains designed miRNA/shRNA constructs targeting almost all human and mouse genes (e.g., Silva et al., 2005, Nature Genetics 37:1281-1288). Any specific member of the library can be readily cloned (such as by PCR) into the vectors of the instant description for Pol II-driven regulated and stable expression.

Expression in some vector designs with different promoters depends on position of transcriptional start and stop sites. The subject method/system described herein, particularly using pol II promoters, has no such stringent requirements.

Another aspect of the description provides a method for drug target validation. The outcome of inhibiting the function of a gene, especially the associated effect in vivo, is usually hard to predict. Gene knock-out experiments offer valuable data for this purpose, but is expensive, time consuming, and potentially non-informative since many genes are required for normal development, such that loss-of-function mutation in such genes causes embryonic lethality. Using the methods of the instant description, especially the inducible expression regulation system of the description, any potential drug target/candidate gene for therapeutic intervention may be tested first by selectively up- and/or down-regulating their expression in vitro, ex vivo, or in vivo, and determining the effect of such regulated expression, especially in vivo effects on an organism. If disruption of the normal expression pattern of a candidate gene shows desired phenotypes in vitro and/or in vivo, the candidate gene is chosen as a target for therapeutic intervention. Various candidate compounds can then be screened to identify inhibitors or activators of such validated targets.

Another aspect of the description provides a method to determine the effect of coordinated expression regulation of two or more genes. For example, miRNA/shRNA constructs for two more target genes may be introduced into a target cell (e.g. by stable integration) or an organism (e.g. by viral vector infection or transgenic techniques), and their expression may be individually or coordinately regulated using the inducible, or constitutive, and/or ubiquitous, or tissue specific or developmental specific promoters according to the instant description. Since different inducible promoters are available, the expression of the two or more target genes may be regulated either in the same or opposite direction (e.g., both up- or down-regulating, or one up one down, etc.). Such experiments can provide useful information regarding, inter alia, genetic interaction between related genes.

In certain embodiments, the nucleic acid constructs encode for modified miRNA that allows highly efficient knockdown of a target gene from a single (retroviral) integration event, thus providing a highly efficient means for certain screening applications. For example, the instant system and methods may be used to test potentially lethal miRNA/shRNAs or synthetic lethal miRNA/shRNAs.

Herein described also is a method to treat certain cancer, especially those cancer overexpressing Ras pathway genes (e.g., Ras itself) and having impaired p53 function, comprising introducing into such cells an active p53 gene or gene product to induce senescence and/or apoptosis, thereby killing the cancer cells, or at least inhibit cancer progression and/or growth.

### MicroRNA and RNAi Design

DNA vectors that express perfect complementary short hairpins RNAs (shRNAs) are commonly used to generate functional siRNAs/mature small RNA triggers of RNAi. (As used herein, shRNA, siRNA and mature small RNA are a unified group of molecules that all function to knockdown expression of target genes, and are used interchangeably in that context.) The efficacy of gene silencing mediated by different shRNA-derived siRNAs may be inconsistent, and a number of short-hairpin RNA expression vectors can trigger an anti-viral interferon response. Moreover, shRNAs are typically processed symmetrically, in that both the functional siRNA strand and its complement strand are incorporated into the RISC complex. Entry of both strands into the RISC can decrease the efficiency of the desired regulation and increase the number of off-target mRNAs that are influenced. In comparison, endogenous microRNA (miRNA) processing and maturation is a fairly efficient process that is not expected to trigger an anti-viral interferon response. This process involves sequential steps that are specified by the information contained in miRNA hairpin and its flanking sequences.

Mature microRNAs (miRNAs) are endogenously encoded ˜22-nt-long RNAs that are generally expressed in a highly tissue- or developmental-stage-specific fashion and that post-transcriptionally regulate target genes. More than ˜800 (mammals) distinct miRNAs have been identified in plants and animals. These small regulatory RNAs are believed to serve important biological functions by three prevailing modes of action: (1) by repressing the translation of target mRNAs, (2) through deadenylation of transcripts, and (3) through cleavage and degradation of mRNAs. In the latter case, miRNAs function analogously to small interfering RNAs (siRNAs), in that they lead to a direct cleavage of the transcript rather than a non-cleavage mediated repression of gene expression. Importantly, miRNAs are expressed in a highly tissue-specific or developmentally regulated manner and this regulation is likely key to their predicted roles in eukaryotic development and differentiation. Analysis of the normal role of miRNAs will be facilitated by techniques that allow the regulated over-expression or inappropriate expression of authentic miRNAs in vivo, whereas the ability to regulate the expression of siRNAs will greatly increase their utility both in cultured cells and in vivo. Thus one can design and express artificial microRNAs based on the features of existing microRNA genes, such as the gene encoding the human miR-30 microRNA. These miR30-based shRNAs have complex folds, and, compared with simpler stem/loop style shRNAs, are less toxic at inhibiting gene expression in transient and long-term assays.

Expression requires the insertion of the entire or core elements of the predicted miRNA precursor stem-loop structure into the expression vector at an arbitrary location. Because the actual extent of the precursor stem loop can sometimes be difficult to accurately predict, it is generally appropriate to include ^{˜}20-150 bp of flanking sequence on each side of the predicted ^{˜}60-80-nt miRNA stem-loop precursor to be sure that all cis-acting sequences necessary for accurate and efficient Drosha processing are included.

Genome wide libraries of shRNAs based on the miR30 precursor RNA have also been generated. Each member of such libraries target specific human or mouse genes (or other species, including rat, dog, cat, monkey, cow, . . . ), and may be readily converted to the vectors/expression systems of the instant description. The following section describes the design of such libraries.

MicroRNAs (including the siRNA/mature small RNA products and artificial microRNAs as well as endogenous microRNAs) have potential for use as therapeutics as well as research tools, e.g. analyzing gene function. As a general method, the mature microRNA (miR) of the description, especially those non-miR-30 based microRNA constructs of the description may also be produced according to the following description.

In certain embodiments, the methods for efficient expression of microRNAs involve the use of a precursor microRNA molecule having a microRNA sequence in the context of microRNA flanking sequences. The precursor microRNA is composed of any type of nucleic acid based molecule capable of accommodating the microRNA flanking sequences and the microRNA sequence. Examples of precursor microRNAs and the individual components of the precursor (flanking sequences and microRNA sequence) are provided herein. The description, however, is not limited to the examples provided. The description is based, at least in part, on the discovery of an important component of precursor microRNAs, that is, the microRNA flanking sequences. The nucleotide sequence of the precursor and its components may vary widely.

In one aspect a precursor microRNA molecule is an isolated nucleic acid including microRNA flanking sequences and having a stem-loop structure with a microRNA sequence incorporated therein. An “isolated molecule” is a molecule that is free of other substances with which it is ordinarily found in nature or in vivo systems to an extent practical and appropriate for its intended use. In particular, the molecular species are sufficiently free from other biological constituents of host cells or if they are expressed in host cells they are free of the form or context in which they are ordinarily found in nature. For instance, a nucleic acid encoding a precursor microRNA having homologous microRNA sequences and flanking sequences may ordinarily be found in a host cell in the context of the host cell genomic DNA. An isolated nucleic acid encoding a microRNA precursor may be delivered to a host cell, but is not found in the same context of the host genomic DNA as the natural system. Alternatively, an isolated nucleic acid is removed from the host cell or present in a host cell that does not ordinarily have such a nucleic acid sequence. Because an isolated molecular species of the description may be admixed with a pharmaceutically-acceptable carrier in a pharmaceutical preparation or delivered to a host cell, the molecular species may comprise only a small percentage by weight of the preparation or cell. The molecular species is nonetheless isolated in that it has been substantially separated from the substances with which it may be associated in living systems.

An “isolated precursor microRNA molecule” is one which is produced from a vector having a nucleic acid encoding the precursor microRNA. Thus, the precursor microRNA produced from the vector may be in a host cell or removed from a host cell. The isolated precursor microRNA may be found within a host cell that is capable of expressing the same precursor. Since the isolated precursor miRNA is a synthetic construct produced from a vector, it may be isolated from the host cell by ordinary methods of the art.

The term “nucleic acid” is used to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars, e.g. locked nucleic acids (LNAs).

“MicroRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure.

Thus, in some embodiments the flanking sequences are 5 to 4,000 nt in length. As a result, the length of the precursor molecule may be, in some instances at least about 50 nt or about 100 nt in length. The total length of the precursor molecule, however, may be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900 4,000 and any integer there between.

The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. Thus, one end (i.e., 5′) of the stem-loop structure may be adjacent to a single flanking sequence and the other end (i.e., 3′) of the stem-loop structure may not be adjacent to a flanking sequence. Preferred structures have flanking sequences on both ends of the stem-loop structure. The flanking sequences may be directly adjacent to one or both ends of the stem-loop structure or may be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). In some cases, the loop may also be very short and thereby not be recognized by Dicer, leading to Dicer-independent shRNAs (comparable to the endogenous miR0451). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the description as long as the secondary structure is present. As is known in the art, the secondary structure does not require exact base-pairing. Thus, the stem may include one or more base mismatches. Alternatively, the base-pairing may be exact, i.e. not include any mismatches.

In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof.

In an alternative embodiment, useful interfering RNAs can be designed with a number of software programs, e.g., the OligoEngine siRNA design tool available at wwv.olioengine.com. The siRNAs of this description may range about, e.g., 19-29 basepairs in length for the double-stranded portion. In some embodiments, the siRNAs are hairpin RNAs having an about 19-29 bp stem and an about 4-34 nucleotide loop. Preferred siRNAs are highly specific for a region of the target gene and may comprise any about 19-29 bp fragment complementary to a target gene mRNA that has at least one, preferably at least two or three, by mismatch with a nontarget gene-related sequence. In some embodiments, the preferred siRNAs do not bind to RNAs having more than 3 mismatches with the target region.

### Expression Vectors and Host Cells

The description also includes vectors for producing precursor microRNA molecules. Generally these vectors include a sequence encoding a primary microRNA and (in vivo) expression elements. The expression elements include at least one promoter, such as a Pol II promoter, which may direct the expression of the operably linked microRNA precursor (e.g. the shRNA encoding sequence). The vector or primary transcript is first processed to produce the stem-loop precursor molecule. The stem-loop precursor is then processed to produce the mature microRNA.

RNA polymerase II (Pol II) transcription units (e.g., units containing a CMV promoter) is preferred for use with inducible expression. It will be appreciated that in the vectors of the description, the subject shRNA encoding sequence may be operably linked to a variety of other promoters. In some embodiments, the promoter is a type II tRNA promoter such as the tRNAVa promoter and the tRNAmet promoter. These promoters may also be modified to increase promoter activity. In addition, enhancers can be placed near the promoter to enhance promoter activity. Pot II enhancer may also be used for Pol III promoters. For example, an enhancer from the CMV promoter can be placed near the U6 promoter to enhance U6 promoter. In certain embodiments, the subject Pol II promoters are inducible promoters. Exemplary inducible Pol II systems are available from commercial sources. Inducible promoters include Tet-responsive promoters, macrolide responsive promoters and the lac operator system.

The interfering RNA can be inducibly expressed in a tissue-specific manner dictated by a tissue-specific promoter driving expression of a tet-transactivator protein (tTA;TET-off or rtTA;TET-on). In certain embodiments, tissue-specificity can be obtained by coupling tissue-specific promoters to the Cre-LoxP system. In general, a triple transgenic can be produced consisting of (1) TRE-Lox-Stop-Lox(LSL)-GFP-miR30/E, (2) CAG-rtTA, and (3) tissue specific promoter-CRE; or (1) TRE-GFP-miR30/E, (2) CAG-LSL-rtTA, and (3) tissue specific promoter-CRE. Tissue-specific promoters that can be used include, without limitation: a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a RIP promoter in the case of pancreatic beta cells; a Keratin promoter in the case of keratinocytes; a Probasin promoter in the case of prostatic epithelium; a Nestin or GFAP promoter in the case of CNS cells and/or cancers; a Tyrosine Hydroxylase, S100 promoter or neurofilament promoter in the case of neurons; the pancreas-specific promoter; a Clara cell secretory protein promoter in the case of lung cancer; and an Alpha myosin promoter in the case of cardiac cells.

Cre and/or tet-transactivator expression also can be controlled in a temporal manner, e.g., by using an inducible promoter, or a promoter that is temporally restricted during development such as Pax3 or Protein 0 (neural crest), Hoxal (floorplate and notochord), Hoxb6 (extraembryonic mesoderm, lateral plate and limb mesoderm and midbrain-hindbrain junction), Nestin (neuronal lineage), GFAP (astrocyte lineage), Lck (immature thymocytes). Temporal control also can be achieved by using an inducible form of Cre. For example, one can use a small molecule controllable Cre fusion, for example a fusion of the Cre protein and the estrogen receptor (ER) or with the progesterone receptor (PR). Tamoxifen or RU486 allow the Cre-ER or Cre-PR fusion, respectively, to enter the nucleus and recombine the LoxP sites, removing the LoxP Stop cassette. Mutated versions of either receptor may also be used. For example, a mutant Cre-PR fusion protein may bind RU486 but not progesterone. Other exemplary Cre fusions are a fusion of the Cre protein and the glucocorticoid receptor (GR). Natural GR ligands include corticosterone, cortisol, and aldosterone. Mutant versions of the GR receptor, which respond to, e.g., dexamethasone, triamcinolone acetonide, and/or RU38486, may also be fused to the Cre protein.

In certain embodiments, additional transcription units may be present 3′ to the shRNA portion. For example, an internal ribosomal entry site (IRES) may be positioned downstream of the shRNA insert, the transcription of which is under the control of a second promoter, such as the PGK promoter. The IRES sequence may be used to direct the expression of an operably linked second gene, such as a reporter gene (e.g., a fluorescent protein such as GFP, BFP, YFP, etc., an enzyme such as luciferase (Promega), etc.). The reporter gene may serve as an indication of infection/transfection, and the efficiency and/or amount of mRNA transcription of the shRNA-IRES-reporter cassette/insert. Optionally, one or more selectable markers (such as puromycin resistance gene, neomycin resistance gene, hygromycin resistance gene, zeocin resistance gene, etc.) may also be present on the same vector, and are under the transcriptional control of the second promoter. Such markers may be useful for selecting stable integration of the vector into a host cell genome.

Certain exemplary vectors useful for expressing the precursor microRNAs are shown in the examples. Thus the description encompasses the nucleotide sequence of such vectors as well as variants thereof.

In general, variants typically will share at least 40% nucleotide identity with any of the described vectors, in some instances, will share at least 50% nucleotide identity; and in still other instances, will share at least 60% nucleotide identity. The preferred variants have at least 70% sequence homology. More preferably the preferred variants have at least 80% and, most preferably, at least 90% sequence homology to the described sequences. (If the common element of the shown vector(s) and the variant vector is only the miR-E backbone of the precursor miRNAs described herein, then the sequence/nucleotide identity will only be ˜300 nt of an ˜6000-9000 nt vector, which is about 3-5%.)

When using the molecules described herein to produce variants from libraries, variants with high percentage sequence homology can be identified, for example, using stringent hybridization conditions. The term “stringent conditions”, as used herein, refers to parameters with which the art is familiar. More specifically, stringent conditions, as used herein, refer to hybridization at 65° C. in hybridization buffer (3.5×SSC, 0.02% Ficoll, 0.02% polyvinyl pyrolidone, 0.02% bovine serum albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetraacetic acid. After hybridization, the membrane to which the DNA is transferred is washed at 2×SSC at room temperature and then at 0.1×SSC/0.1×SDS at 65° C. There are other conditions, reagents, and so forth which can be used, which result in a similar degree of stringency. Such variants may be further subject to functional testing such that variants that substantially preserve the desired/relevant function of the original vectors are selected/identified.

The “in vivo expression elements” are any regulatory nucleotide sequence, such as a promoter sequence or promoter-enhancer combination, which facilitates the efficient expression of the nucleic acid to produce the precursor microRNA. The in vivo expression element may, for example, be a mammalian or viral promoter, such as a constitutive or inducible promoter or a tissue specific promoter. Constitutive mammalian promoters include, but are not limited to, polymerase II promoters as well as the promoters for the following genes: hypoxanthine phosphoribosyl transferase (HPTR), adenosine deaminase, pyruvate kinase, and β-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as in vivo expression element of the description also include inducible promoters. Inducible promoters are expressed in the presence of an inducing agent. For example, the metallothionein promoter is induced to promote transcription in the presence of certain metal ions. Other inducible promoters are known to those of ordinary skill in the art.

Vectors include, but are not limited to, plasmids, phagemids, viruses, other vehicles derived from viral or bacterial sources that have been manipulated by the insertion or incorporation of the nucleic acid sequences for producing the precursor microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; lentiviruses; and RNA viruses such as any retrovirus. One can readily employ other unnamed vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Clifton, N.J. (1991).

The description also encompasses host cells transfected with the subject vectors, especially host cell lines with stably integrated shRNA constructs. In certain embodiments, the subject host cell contains only a single copy of the integrated construct expressing the desired shRNA (optionally under the control of an inducible and/or tissue specific promoter). Host cells include for instance, cells (such as primary cells) and cell lines, e.g. prokaryotic (e.g., E. coli), and eukaryotic (e.g., dendritic cells, CHO cells, COS cells, yeast expression systems and recombinant baculovirus expression in insect cells, etc.). Exemplary cells include: NIH3T3 cells, MEFs, HEK293 or HEK293T cells, CHO cells, DF1 cells, hematopoietic stem/progenitor cells, cancer cells, etc.

### Methods of Using

In certain aspects, methods of the description comprise contacting and introducing into a target cell with a subject vector capable of expressing a precursor microRNA as described herein, to regulate the expression of a target gene in the cell. The vector produces the microRNA transcript, which is then processed into precursor microRNA in the cell, which is then processed to produce the mature functional microRNA which is capable of altering accumulation of a target protein in the target cell. Accumulation of the protein may be effected in a number of different ways. For instance the microRNA may directly or indirectly affect translation or may result in cleavage of the mRNA transcript or even effect stability of the protein being translated from the target mRNA. MicroRNA may function through a number of different mechanisms. The methods and products of the description are not limited to any one mechanism. The method may be performed in vitro, e.g., for studying gene function, ex vivo or in vivo, e.g. for therapeutic purposes.

An “ex vivo” method as used herein is a method which involves isolation of a cell from a subject, manipulation of the cell outside of the body, and reimplantation of the manipulated cell into the subject. The ex vivo procedure may be used on autologous or heterologous cells, but is preferably used on autologous cells. In preferred embodiments, the ex vivo method is performed on cells that are isolated from bodily fluids such as peripheral blood or bone marrow, but may be isolated from any source of cells. When returned to the subject, the manipulated cell will be programmed for cell death or division, depending on the treatment to which it was exposed. Ex vivo manipulation of cells has been described in several references in the art. The ex vivo activation of cells of the description may be performed by routine ex vivo manipulation steps known in the art. In vivo methods are also well known in the art. The description thus is useful for therapeutic purposes and also is useful for research purposes such as testing in animal or in vitro models of medical, physiological or metabolic pathways or conditions.

The ex vivo and in vivo methods are performed on a subject. A “subject” shall mean a human or non-human mammal, including but not limited to, a dog, cat, horse, cow, pig, sheep, goat, primate, rat, and mouse, etc. In other instances, the “subject” may also be a non-mammal vertebrate, including but not limited to chicken, frog, fish, etc.

In some instances the mature microRNA is expressed at a level sufficient to cause at least a 2-fold, or in some instances, a 10 fold reduction in accumulation of the target protein. The level of accumulation of a target protein may be assessed using routine methods known to those of skill in the art. For instance, protein may be isolated from a target cell and quantitated using Western blot analysis or other comparable methodologies, optionally in comparison to a control. Protein levels may also be assessed using reporter systems or fluorescently labeled antibodies. In other embodiments, the mature microRNA is expressed at a level sufficient to cause at least a 2, 5, 15, 20, 25, 30, 35, 40, 45, 50, 55, 60, 65, 70, 75, or 100 fold reduction in accumulation of the target protein. The “fold reduction” may be assessed using any parameter for assessing a quantitative value of protein expression. For instance, a quantitative value can be determined using a label i.e. fluorescent, radioactive linked to an antibody. The value is a relative value that is compared to a control or a known value.

Different microRNA sequences have different levels of expression of mature microRNA and thus have different effects on target mRNA and/or protein expression. For instance, in some cases a microRNA may be expressed at a high level and may be very efficient such that the accumulation of the target protein is completely or near completely blocked. In other instances the accumulation of the target protein may be only reduced slightly over the level that would ordinarily be expressed in that cell at that time under those conditions in the absence of the mature microRNA. Complete inhibition of the accumulation of the target protein is not essential, for example, for therapeutic purposes. In many cases partial or low inhibition of accumulation may produce a preferred phenotype. The actual amount that is useful will depend on the particular cell. type, the stage of differentiation, conditions to which the cell is exposed, the modulation of other target proteins, etc.

The microRNAs may be used to knock down gene expression in vertebrate cells for gene-function studies, including target-validation studies during the development of new pharmaceuticals, as well as the development of human disease models and therapies, and ultimately, human gene therapies.

The methods of the description are useful for developing therapies for any type of “disease”, “disorder” or “condition” in which it is desirable to reduce the expression or accumulation of a particular target protein(s). Diseases include, for instance, but are not limited to, cancer, infectious disease, cystic fibrosis, blood disorders, including leukemia and lymphoma, spinal muscular dystrophy, early-onset Parkinsonism (Waisman syndrome) and X-linked mental retardation (MRx3).

Cancers include but are not limited to biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

An infectious disease, as used herein, is a disease arising from the presence of a foreign microorganism in the body. A microbial antigen, as used herein, is an antigen of a microorganism. Microorganisms include but are not limited to, infectious virus, infectious bacteria, and infectious fungi.

Examples of infectious virus include but are not limited to: Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema palladium, Treponema pertenue, Leptospira, Rickettsia, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium such as Plasmodium falciparum, Plasmodium malariae, Plasmodium ovale, and Plasmodium vivax and Toxoplasma gondii.

The vectors of this description can be delivered into host cells via a variety of methods, including but not limited to, liposome fusion, infection by viral vectors, and routine nucleic acid transfection methods such as electroporation, calcium phosphate precipitation and microinjection. In some embodiments, the vectors are integrated into the genome of a transgenic animal (e.g., a mouse, a rabbit, a hamster, or a nonhuman primate). Diseased or disease-prone cells containing these vectors can be used as a model system to study the development, maintenance, or progression of a disease that is affected by the presence or absence of the interfering RNA.

Expression of the miRNA/siRNA introduced into a target cell may be confirmed by art-recognized techniques, such as Northern blotting using a nucleic acid probe. For cell lines that are more difficult to transfect, more extracted RNA can be used for analyses, optionally coupled with exposing the film longer. Once expression of the miRNA/siRNA is confirmed, the DNA construct can then be tested for RNAi efficacy against a cotransfected construct encoding the target protein or directly against an endogenous target. In the latter case, one preferably should have a clear idea of transfection efficiency and of the half-life of the target protein before performing the experiment.

### Pharmaceutical Use and Methods of Administration

In one aspect, the description provides a method of administering any of the compositions described herein to a subject. When administered, the compositions are applied in a therapeutically effective, pharmaceutically acceptable amount as a pharmaceutically acceptable formulation. As used herein, the term “pharmaceutically acceptable” is given its ordinary meaning. Pharmaceutically acceptable compounds are generally compatible with other materials of the formulation and are not generally deleterious to the subject. Any of the compositions of the present description may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result, i.e., that amount which is capable of at least partially preventing, reversing, reducing, decreasing, ameliorating, or otherwise suppressing the particular condition being treated. A therapeutically effective amount can be determined on an individual basis and will be based, at least in part, on consideration of the species of mammal, the mammal's age, sex, size, and health; the compound and/or composition used, the type of delivery system used; the time of administration relative to the severity of the disease; and whether a single, multiple, or controlled-release dose regiment is employed. A therapeutically effective amount can be determined by one of ordinary skill in the art employing such factors and using no more than routine experimentation.

The terms “treat,” “treated,” “treating,” and the like, when used herein, refer to administration of the systems and methods of the description to a subject, which may, for example, increase the resistance of the subject to development or further development of cancers, to administration of the composition in order to eliminate or at least control a cancer or an infectious disease, and/or to reduce the severity of the cancer or infectious disease, or symptoms thereof. Such terms also include prevention of disease/condition in, for example, subjects/individuals predisposed to such diseases/conditions, or at high risk of developing such diseases/conditions.

When administered to a subject, effective amounts will depend on the particular condition being treated and the desired outcome. A therapeutically effective dose may be determined by those of ordinary skill in the art, for instance, employing factors such as those further described below and using no more than routine experimentation.

### Exemplary Uses

Drug Target Validation

Good drugs are potent and specific; that is, ideally, they must have strong effects on a specific biological pathway or tissue (such as the disease tissue), while having minimal effects on all other pathways or all other tissues (e.g., healthy tissues). Confirmation that a compound inhibits the intended target (drug target validation) and the identification of undesirable secondary effects are among the main challenges in developing new drugs. Drug target validation also includes determining whether the inhibition of the target under consideration has a therapeutic benefit.

Modern drug screening typically requires tremendous amounts of time and financial resources. Ideally, before even committing to such an extensive drug development program to identify a drug, one would like to know whether the intended drug target would even make a good target for treating a disease. That is, whether antagonizing the function of the intended target (such as a disease-associated oncogene or survival gene), would be sufficient/effective to treat the disease, and whether such treatment would bear an acceptable risk or side effect. For example, if a cancer is determined to be caused by an activating mutation in the Ras pathway, or caused by abnormal activity of a survival gene such as Bc1-2, the subject system can be used to generate animal models for drug target validation. Specifically, one can generate a transgenic mouse with the subject tet-responsive miRNA-embedded shRNA expression, with the miRNA-embedded shRNA targeting a gene that is a potential drug target (i.e., Ras or Bc1-2 in this example). Tumors with various initiating lesions can then be made in the mouse, and the miRNA-embedded shRNA can be switched on in the tumor (if, for example, a tet-ON regulator is used). Such miRNA-embedded shRNA expression mimics the action of a (yet to be identified) drug that would interfere with that target. If knocking down the target gene is effective to reverse or stall the course of the disease, the target gene is a valid target.

Optionally, the miRNA-embedded shRNA transgene can be switched on in a number of tissues or organs, or even in the whole organism, in order to verify the potential side effects of the (yet to be identified) drug on other healthy tissues/organs.

Thus another aspect of the description provides an animal useful for drug target validation, comprising a germline transgene encompassing the subject artificial nucleic acid, which transcription is driven by a Pol II promoter. The expression of the encoded precursor molecule (such as one based on the miR30-design) leads to an siRNA/mature small RNA trigger of RNAi that targets a (yet to be identified) candidate drug target. Optionally, the precursor molecule is expressed in an inducible, reversible, and/or tissue-specific manner.

In a related aspect, the description provides a method for drug target validation, comprising antagonizing the function of a candidate drug target (gene) using a subject cell or animal (e.g., a transgenic animal) encompassing the subject artificial nucleic acid, either in vitro or in vivo, and assessing the ability of the encoded precursor molecule to reverse or stall the disease progress or a particular phenotype associated with a pathological condition. Optionally, the method further comprises assessing any side effects of inhibiting the function of the target gene on one or more healthy organs/tissues.

Animal Disease Model

The subject nucleic acid constructs enables one to switch on or off a target gene or certain target genes (e.g., by using crossing different lines of transgenic animals to generate multiple-transgenic animals) inducibly, reversibly, and/or in a tissue-specific manner. This would facilitate conditional knock-out/knock-down or turning-on of any target gene(s) in a tissue-specific manner, and/or during a specific developmental stage (e.g., embryonic, fetal, neonatal, postnatal, adult, etc.). Animals bearing such transgenes may be treated, such as by providing a tet analog in drinking water, to turn on or off certain genes to allow certain diseases to develop/manifest. Such system and methods are particularly useful, for example, to analyze the role of any known or suspected oncogene (or tumor suppressor genes) in the maintenance of immortalized or transformed states, and in continued tumor growth in vivo.

In certain embodiments, the extent of gene knock-down may be controlled to achieve a desired level of gene expression. Such animals or cell (healthy or diseased) may be used to study disease progress, response to certain treatment, and/or screening for drug leads.

The ability of the subject system to use a single genomic copy of the Pol II promoter-driven miRNA-embedded shRNA cassette to control gene expression is particularly valuable for complex library screening.

The subject gene knock-down by expression of shRNA-mirs may be very similar to overexpression of protein-coding cDNAs. Thus any expression systems allowing targeted, regulated and tissue-specific expression, which have traditionally be limited to gene overexpression studies, may now be adapted for loss-of-function studies, especially when combined with the available genome-wide, sequence-verified banks of miR-30-based shRNAs targeting model organisms, such as human and mouse.

### EXAMPLES

### Example 1

### Conserved Region

Comparison of miR-30 sequence across several species shows an evolutionary conserved region (FIG. 1). The alignment shows a highly conserved region extends from reference position 239 to 340. A comparison of complete sequence of endogenous and synthetic miR-30 shows that the guide/passenger positioning is reversed in synthetic miR-30, and that a bulge in position 12-13 of the guide is missing. Importantly, a synthetic loop in a 3′ conserved region contains mutations in one highly conserved nucleotide. This is because a EcoRI site is placed there, and alters a highly conserved nucleotide, i.e., a C is changed to A.

Modifications were made in the conserved region by modifying the vector sequence encoding the miR-30 sequence 5′-GAAUUCAA-3′ to determine the effect on the efficiency of knockdown. The following sequence was used in the vector sh.Yap1.891, noting that that vector contains the DNA sequence 5′-GAATTCAA-3′ where underscored.

(SEQ ID NO: 1)
TGTTTGAATGAGGCTTCAGTACTTTACAGAATCGTTGCCTGCACATCTTG
GAAACACTTGCTGGGATTACTTCTTCAGGTTAACCCAACAGAAGGCTCGA
GAAGGTATATTGCTGTTGACAGTGAGCGCTGGAGAAGTTTACTACATAAA
TAGTGAAGCCACAGATGTATTTATGTAGTAAACTTCTCCATTGCCTACTG
CCTCGGAATTCAAGGGGCTACTTTAGGAGCAATTATCTTGTTTACTAAAA
CTGAATACCTTGCTATCTCTTTGATACATTTTTACAAAGCTGAATTAAAA
TGGTATAAATTAAATCACTTT

The following sequence was used in the vector miR-E sh.Yap1.891, noting that that vector contains the conserved DNA sequence 5′-GACTTCAA-3′ where underscored.

(SEQ ID NO: 2)
TGTTTGAATGAGGCTTCAGTACTTTACAGAATCGTTGCCTGCACATCTTG
GAAACACTTGCTGGGATTACTTCGACTTCTTAACCCAACAGAAGGCTCGA
GAAGGTATATTGCTGTTGACAGTGAGCGCTGGAGAAGTTTACTACATAAA
TAGTGAAGCCACAGATGTATTTATGTAGTAAACTTCTCCATTGCCTACTG
CCTCGGACTTCAAGGGGCTAGAATTOGAGCAATTATCTTGTTTACTAAAA
CTGAATACCTTGCTATCTCTTTGATACATTTTTACAAAGCTGAATTAAAA
TGGTATAAATTAAATCACTTT

The sequence contains a shifted EcoRI site (GAATTC) in a nonconserved region and modifications to preserve the secondary structure in the region predicted to hybridize with the EcoRI site in the miRNA secondary structure.

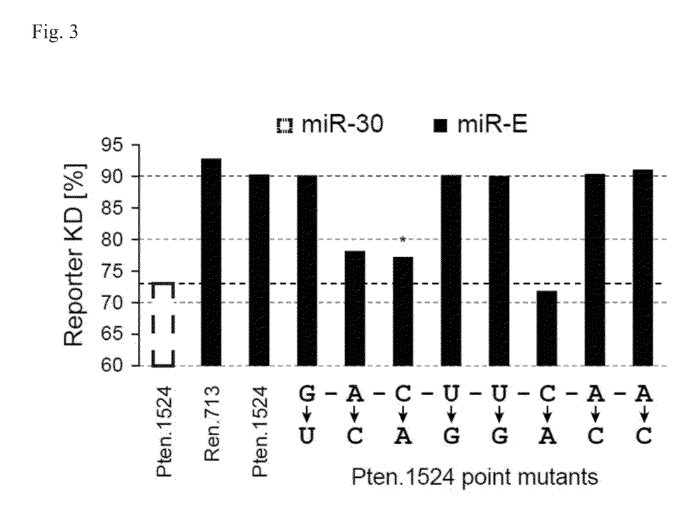

The results (FIG. 3) show that single nucleotide substitutions to produce the miRNA sequences 5′-UACUUCAA-3′, and 5′-GACGUCAA-3′ produced the knockdown equivalent to the endogenous human miR-30 sequence (5′-GACUUCAA-3′). These effects are due to the presence of the original conserved 3′ flank, not the synthetic EcoRI site of miR-E.

The results show that there are critical nucleotides in this region. The perfect configuration was found to be 5′-NACNTCAA-3′. These results show that the modified miRNA-backbone must contain the native sequence 5′-GACUUCAA-3′ or a modified sequence 5′-UACUUCAA-3′, or 5′-GACGUCAA-3′ to maximize knockdown of the embedded shRNA.

### Example 2

### Stem Loop Region

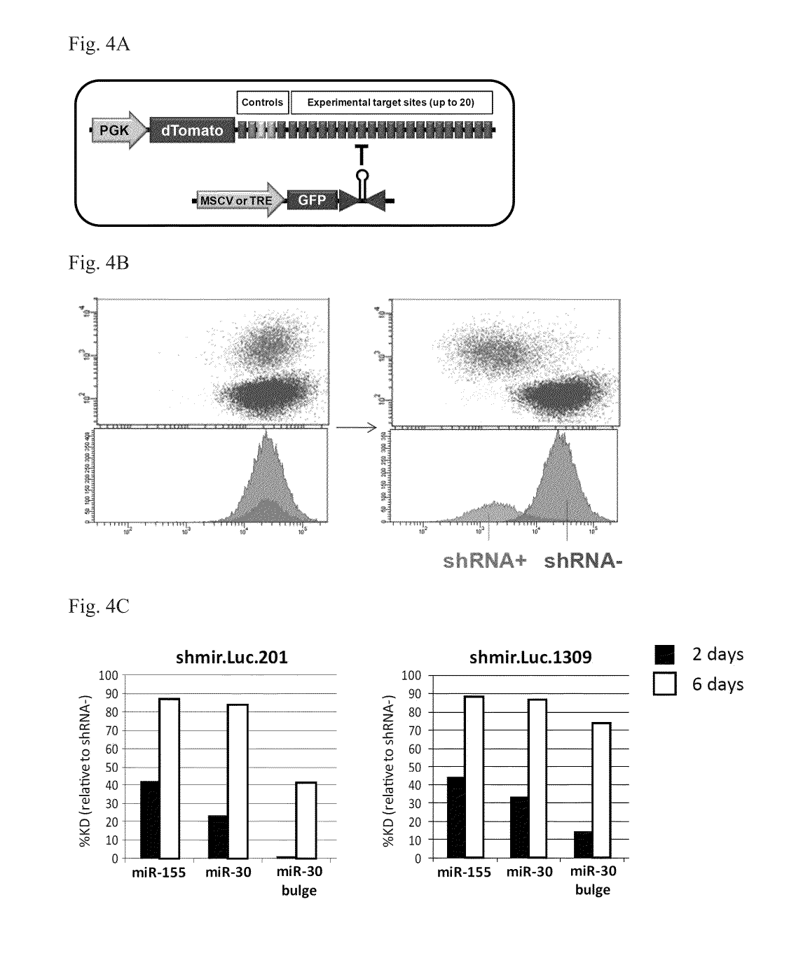

Modifications were made in the stem loop region of miR-30. The miR-30 molecule without and with a bulge was compared to miR-155 (BLOCK-iT, Invitrogen), which has a guide bulge. Two validated Luc shmiR molecules were tested, shmir.Luc.1309, originally validated in miR-30; and shmir Luc.201, a control shRNA from Invitrogen. All were cloned in pLMN and infected at identical single-copy conditions. A sensor shRNA reporter assay system was used according to Fellmann et al., 2011, Mol Cell 41:733-46, hereby incorporated by reference, with some modifications. The experimental design and results are shown in FIGS. 4A, 4B, and 4C. Expression at day 2 and day 6 for each nucleic acid construct were measured and compared. The results show that a bulge in miR-30 guide strand at position 12-13 reduces knockdown.

### Example 3

### Alternative miR-30 Loops

Modifications were made in the miR-30 loops. Modifying the loop during testing of the original guide/passenger configuration did not affect knockdown potential. Seven out of seven alternative synthetic loops turned out to be worse than the miR-E/miR-30 loop. According to Gu et al., 2012, Cell 151:900-11, hereby incorporated by reference, the current configuration perfectly supports precise DICER positioning and minimizes off-target effects. The results show that the currently used miR-30-loop leads to efficient processing in the nucleus and by Dicer.

### Example 4

### Reversing Guide and Passenger Strands in the Stem Loop Region

Modifications were made in the stem loop region to determine whether reversing the guide and passenger strands affects the degree of knockdown achieved. A miRNA-embedded shRNA for knocking down expression of the phosphatase and tensin homolog gene (Pten) was used (FIG. 5A). miR-30 sh.Pten.1523 contained the guide on the 3′ strand connected via a 19 nucleotide loop to the passenger strand on the 5′ loop, as follows.

(SEQ ID NO: 3)
CTCGAGAAGGTATATTGCTGTTGACAGTGAGCGACCAGCTAAAGGTGAA
GATATATAGTGAAGCCACAGATGTATATATCTTCACCTTTAGCTGGCTG
CCTACTGCCTCGGAATTC

miR-30 sh.Pten.1524 contained a different guide and passenger strand, as follows.

(SEQ ID NO: 4)
CTCGAGAAGGTATATTGCTGTTGACAGTGAGCGACAGCTAAAGGTGAAG
ATATATTAGTGAAGCCACAGATGTAATATATCTTCACCTTTAGCTGGTG
CCTACTGCCTCGGAATTC

miR-G5 sh.Pten.1523 and sh.Pten.1524 both contained the guide on the 5′ strand connected via a loop to the passenger strand on the 3′ loop. The sequence of miR-G5 sh.Yap1.891 is as follows.

(SEQ ID NO: 5)
TGTTTGAATGAGGCTTCAGTACTTTACAGAATCGTTGCCTGCACATCTT
GGAAACACTTGCTGGGATTACTTCTTCAGGTTAACCCAACAGAAGGCTC
GAGAAGGTATATTGCTGTTGACAGTGAGCGACTTTATGTAGTAAACTTC
TCCATGTGAAGCCACAGATGATGGAGAAGTTTACTACATAAAGCTGCCT
ACTGCCTCGGAATTCAAGGGGCTACTTTAGGAGCAATTATCTTGTTTAC
TAAAACTGAATACCTTGCTATCTCTTTGATACATTTTTACAAAGCTGAA
TTAAAATGGTATAAATTAAATCACTTT

The core (XhoI/EcoRI) sequence of miR-G5 sh.Pten.1523 is as follows.

(SEQ ID NO: 6)
CTCGAGAAGGTATATTGCTGTTGACAGTGAGCGACTATATCTTCACCTT
TAGCTGGCGTGAAGCCACAGATGGCCAGCTAAAGGTGAAGATATAGCTG
CCTACTGCCTCGGAATTC

The core (XhoI/EcoRI) sequence of miR-G5 sh.Pten.1524 is as follows.

(SEQ ID NO: 7)
CTCGAGAAGGTATATTGCTGTTGACAGTGAGCGACATATATCTTCACCT
TTAGCTGGGTGAAGCCACAGATGCCAGCTAAAGGTGAAGATATATGCTG
CCTACTGCCTCGGAATTC

The sequence encoding modified miR-30 (miR-G5) with these shRNAs (sh.Pten.2634.sh.Pten.1524) was inserted into a LMT vector (LMP with turbo-GFP instead of GFP). A small portion of the sequence of LMP miR-30, around the miRNA-embedded shRNA is as follows.

(SEQ ID NO: 8)
CCTGGGAAGCCTTGGCTTTTGACCCCCCTCCCTGGGTCAAGCCCTTTGT
ACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCC
TCACTCCTTCTCTAGGCGCCGGAATTAGATCTCTCGACTAGGGATAACA
GGGTAATTGTTTGAATGAGGCTTCAGTACTTTACAGAATCGTTGCCTGC
ACATCTTGGAAACACTTGCTGGGATTACTTCTTCAGGTTAACCCAACAG
AAGGCTCGAGCAACCAGAATTCAAGGGGCTACTTTAGGAGCAATTATCT
TGTTTACTAAAACTGAATACCTTGCTATCTCTTTGATACATTTTTACAA
AGCTGAATTAAAATGGTATAAATTAAATCACTTTTTTCAATTGACGCGT
GATCTAATTCTACCGGGTAGGGGAGGCGCTTTTCCCAAGGCAGTCTGGA
GCATGCGCTTTAGCAGCCCCGCTGGGCACTTGGCGCTACACAAGTGGCC
TCTGGCCTCGCACACATTCCACATCCACCGGTAGGCGCCAACCGGCTCC
GT

NIH3T3 full cell lysates in single copy infection were used, and the transfectants were selected by puromycin. The results showed (FIG. 5B) that reversing the guide and passenger strands had no major effects on knockdown potency of miR30-shRNAs.

### Example 5

### Compiling Modifications to Produce miR-E

A modified miR-30 molecule was prepared by correcting the synthetic molecule to restore the endogenous human MIR30A sequence, by including a EcoRI site in a nonconserved region. The resulting nucleic acid sequence of the new molecule, miR-E (with a stem sequence coding for an shRNA targeting the Yap1 gene), is as follows (restriction sites for XhoI 5′-CTCGAG-3′ and EcoRI 5′-GAATTC-3′ are shown in italic):

(SEQ ID NO: 2)
TGTTTGAATGAGGCTTCAGTACTTTACAGAATCGTTGCCT
GCACATCTTGGAAACACTTGCTGGGATTACTTCGACTTCT
TAACCCAACAGAAGGCTCGAGAAGGTATATTGCTGTTGAC
AGTGAGCGCTGGAGAAGTTTACTACATAAATAGTGAAGCC
ACAGATGTATTTATGTAGTAAACTTCTCCATTGCCTACTG
CCTCGGACTTCAAGGGGCTAGAATTCGAGCAATTATCTTG
TTTACTAAAACTGAATACCTTGCTATCTCTTTGATACATT
TTTACAAAGCTGAATTAAAATGGTATAAATTAAATCACTT
T

This miR-E sequence was modified and included shRNA directed to target Pten mRNA. The sequences of these shRNAs (XhoI/EcoRI fragments) are shown in Table 1.

The miR-E encoding vector was transduced into NIH3T3 cells at single copy infection, and the transfectants were selected by puromycin. The results showed (FIG. 6) that the miR-E configuration substantially enhanced Pten knockdown, particularly for intermediate shRNAs.

The structure of miR-E was compared to endogenous miR-30 (MIR30A) and synthetic miR-30, as folded with the mfold algorithm (FIG. 2CB). The conserved region is shown with an arrow, and the non-conserved region circled. The EcoRI site in a conserved region of miR-30 is moved to a nonconserved region in miR-E. The structure shows that the secondary structure of miR-E in the nonconserved region containing the EcoRI site is preserved compared to synthetic and endogenous miR-30.

### Example 6

### General Utility of miR-E

Synthetic miR-30 was compared to miR-E for expression of a variety of siRNA/mature small RNA molecules. Systematic single-copy knockdown testing of a panel of shRNAs (designed based on Sensor rules) in miR-E vs. miR-30 configurations using western blot and/or Sensor-reporter assays (details see above reference to Fellmann et al., 2011, Mol Cell 41:733-46). Primers used to clone shRNAs into miR-E were miR30-Xho-fw (miR30-XhoI-short-fw; 24 bp, Tm 63° C., GC 45%)

(SEQ ID NO: 9)
5′-AGAAGGCTCGAGAAGGTATATTGC-3′
and
miRE-Eco-rev (30 bp, Tm 72° C., GC 56%)
(SEQ ID NO: 10)
5′-GCTCGAATTCTAGCCCCTTGAAGTCCGAGG-3′

The sequences used in comparison are shown in Tables 1 and 2.

The results (FIGS. 8 and 9B) show that the miR-E configuration dramatically enhances knockdown for almost every shRNA. Only known super-shRNAs (e.g. p53.1224, Ren.713) are not further improved, likely because the knockdown is already saturated, while many intermediate shRNAs in miR-E trigger knockdown in the range of super-potent shRNAs. The results show that combining Sensor-based design rules and miR-E configuration produces potent shRNAs in >70%.

### Example 7

### Production of miR-E Virions

PlatGP packaging cells were first stably transduced with stem-loop DGCR8 shRNAs (A-G) and selected. Two shRNAs were lethal. Others were then transfected with the Sensor vector (see legend for FIG. 10). In the presence of the target site (TS) for the miRNA-embedded shRNA expressed from the same plasmid, the Sensor vector dramatically reduces its own viral titers, which can be partially rescued with DGCR8 siRNA cotransfection. One of the five stable DGCR8 knockdown packaging lines shows full restoration of packaging efficacies indicating that pri-miRNA processing is sufficiently blocked (FIG. 10). These packaging cell lines will not only be valuable for even packaging of Sensor constructs or miR-E based shRNAs, but also to correct shRNA-processing induced biases in complex shRNA pools.

TABLE 1
shRNA sequences

| | | | SEQ |
|---|---|---|---|
| | | | ID |
| Backbone | shRNA name | Sequence | NO: |
| miR-30 | sh.Pten.1523 | ctcgagaaggtatattgctgttgac agtgagcgaccagctaaaggtgaag | 11 |
| | | atatatagtgaagccacagatgtat atatcttcacctttagctggctgcc | |
| | | tactgcctcggaattc | |
| miR-30 | sh.Pten.1524 | ctcgagaaggtatattgctgttgac agtgagcgacagctaaaggtgaaga | 12 |
| | | tatattagtgaagccacagatgtaa tatatcttcacctttagctggtgcc | |
| | | tactgcctcggaattc | |
| miR-30 | sh.Ren.713 | ctcgagaaggtatattgctgttgac agtgagcgcaggaattataatgctt | 13 |
| | | atctatagtgaagccacagatgtat agataagcattataattcctatgcc | |
| | | tactgcctcggaattc | |
| miR-30 | sh.Pten.932 | ctcgagaaggtatattgctgttgac agtgagcgccgacttagacttgacc | 14 |
| | | tatattagtgaagccacagatgtaa tataggtcaagtctaagtcgatgcc | |
| | | tactgcctcggaattc | |
| miR-30 | sh.Pten.1688 | ctcgagaaggtatattgctgttgac agtgagcgcttgggtaaatacgttc | 15 |
| | | ttcattagtgaagccacagatgtaa tgaagaacgtatttacccaaatgcc | |
| | | tactgcctcggaattc | |
| miR-30 | sh.Pten.2049 | ctcgagaaggtatattgctgttgac agtgagcgaaagatcagcattcaca | 16 |
| | | aattatagtgaagccacagatgtat aatttgtgaatgctgatcttctgcc | |
| | | tactgcctcggaattc | |
| miR-30 | sh.Bcl2.783 | ctcgagaaggtatattgctgttgac agtgagcgattatacaaggagactt | 17 |
| | | ctgaatagtgaagccacagatgtat tcagaagtctccttgtataagtgcc | |
| | | tactgcctcggaattc | |
| miR-30 | sh.Bcl2.851 | ctcgagaaggtatattgctgttgac agtgagcgcaagggtaaacttgaca | 18 |
| | | gaagatagtgaagccacagatgtat cttctgtcaagtttacccttttgcc | |
| | | tactgcctcggaattc | |
| miR-30 | sh.Bcl2.906 | ctcgagaaggtatattgctgttgac agtgagcgcgcacaggaatttt tt | 19 |
| | | taatatagtgaagccacagatgtat attaaacaaaattcctgtgcatgcc | |
| | | tactgcctcggaattc | |
| miR-30 | sh.Bcl2.1132 | ctcgagaaggtatattgctgttgac agtgagcgcgactgatattaacaaa | 20 |
| | | gcttatagtgaagccacagatgtat aagcttt ttaatatcagtcttgcc | |
| | | tactgcctcggaattc | |
| miR-30 | sh.Bcl2.1241 | ctcgagaaggtatattgctgttgac agtgagcgccaagtgttcggtgtaa | 21 |
| | | ctaaatagtgaagccacagatgtat ttagttacaccgaacacttgatgcc | |
| | | tactgcctcggaattc | |
| miR-30 | sh.Mcl1.772 | ctcgagaaggtatattgctgttgac agtgagcgactccggaaactggaca | 22 |
| | | ttaaatagtgaagccacagatgtat ttaatgtccagtttccggagctgcc | |
| | | tactgcctcggaattc | |
| miR-30 | sh.Mcl1.866 | ctcgagaaggtatattgctgttgac agtgagcgcggattgtgactcttat | 23 |
| | | ttctttagtgaagccacagatgtaa agaaataagagtcacaatccttgcc | |
| | | tactgcctcggaattc | |
| miR-30 | sh.Mcl1.1334 | ctcgagaaggtatattgctgttgac agtgagcgaaagagtcactgtctga | 24 |
| | | atgaatagtgaagccacagatgtat tcattcagacagtgactcttctgcc | |
| | | tactgcctcggaattc | |
| miR-30 | sh.Mcl1.1792 | ctcgagaaggtatattgctgttgaca gtgagcgaaacagcctcgatttttaa | 25 |
| | | gaatagtgaagccacagatgtattct taaaaatcgaggctgttctgcctact | |
| | | gcctcggaattc | |
| miR-30 | sh.Mcl1.2018 | ctcgagaaggtatattgctgttgaca gtgagcgcggactggttatagattta | 26 |
| | | taatagtgaagccacagatgtattat aaatctataaccagtccatgcctact | |
| | | gcctcggaattc | |
| miR-E | sh.Pten.1523 | ctcgagaaggtatattgctgttgaca gtgagcgaccagctaaaggtgaagat | 27 |
| | | atatagtgaagccacagatgtatata tcttcacctttagctggctgcctact | |
| | | gcctcggacttcaaggggctagaatt c | |
| miR-E | sh.Pten.1524 | ctcgagaaggtatattgctgttgaca gtgagcgacagctaaaggtgaagata | 28 |
| | | tattagtgaagccacagatgtaatat atcttcacctttagctggtgcctact | |
| | | gcctcggacttcaaggggctagaatt c | |
| miR-E | sh.Ren.713 | ctcgagaaggtatattgctgttgaca gtgagcgcaggaattataatgcttat | 29 |
| | | ctatagtgaagccacagatgtataga taagcattataattcctatgcctact | |
| | | gcctcggacttcaaggggctagaatt c | |
| miR-E | sh.Pten.932 | ctcgagaaggtatattgctgttgaca gtgagcgccgacttagacttgaccta | 30 |
| | | tattagtgaagccacagatgtaatat aggtcaagtctaagtcgatgcctact | |
| | | gcctcggacttcaaggggctagaatt c | |
| miR-E | sh.Pten.1688 | ctcgagaaggtatattgctgttgaca gtgagcgcttgggtaaatacgttctt | 31 |
| | | cattagtgaagccacagatgtaatga agaacgtatttacccaaatgcctact | |
| | | gcctcggacttcaaggggctagaatt c | |
| miR-E | sh.Pten.2049 | ctcgagaaggtatattgctgttgaca gtgagcgaaagatcagcattcacaaa | 32 |
| | | ttatagtgaagccacagatgtataat ttgtgaatgctgatcttctgcctact | |
| | | gcctcggacttcaaggggctagaatt c | |
| miR-E | sh.Bcl2.783 | ctcgagaaggtatattgctgttgaca gtgagcgattatacaaggagacttct | 33 |
| | | gaatagtgaagccacagatgtattca gaagtctccttgtataagtgcctact | |
| | | gcctcggacttcaaggggctagaatt c | |
| miR-E | sh.Bcl2.851 | ctcgagaaggtatattgctgttgaca gtgagcgcaagggtaaacttgacaga | 34 |
| | | agatagtgaagccacagatgtatctt ctgtcaagtttacccttttgcctact | |
| | | gcctcggacttcaaggggctagaatt c | |
| miR-E | sh.Bcl2.906 | ctcgagaaggtatattgctgttgaca gtgagcgcgcacaggaattttgttta | 35 |
| | | atatagtgaagccacagatgtatatt aaacaaaattcctgtgcatgcctact | |
| | | gcctcggacttcaaggggctagaatt c | |
| miR-E | sh.Bcl2.1132 | ctcgagaaggtatattgctgttgaca gtgagcgcgactgatattaacaaagc | 36 |
| | | ttatagtgaagccacagatgtataag ctttgttaatatcagtcttgcctact | |
| | | gcctcggacttcaaggggctagaatt c | |
| miR-E | sh.Bcl2.1241 | ctcgagaaggtatattgctgttgaca gtgagcgccaagtgttcggtgtaact | 37 |
| | | aaatagtgaagccacagatgtattta gttacaccgaacacttgatgcctact | |
| | | gcctcggacttcaaggggctagaatt c | |
| miR-E | sh.Mcl1.772 | ctcgagaaggtatattgctgttgaca gtgagcgactccggaaactggacatt | 38 |
| | | aaatagtgaagccacagatgtattta atgtccagtttccggagctgcctact | |
| | | gcctcggacttcaaggggctagaatt c | |
| miR-E | sh.Mcl1.866 | ctcgagaaggtatattgctgttgaca gtgagcgcggattgtgactcttattt | 39 |
| | | ctttagtgaagccacagatgtaaaga aataagagtcacaatccttgcctact | |
| | | gcctcggacttcaaggggctagaatt c | |
| miR-E | sh.Mcl1.1334 | ctcgagaaggtatattgctgttgaca gtgagcgaaagagtcactgtctgaat | 40 |
| | | gaatagtgaagccacagatgtattca ttcagacagtgactcttctgcctact | |
| | | gcctcggacttcaaggggctagaatt c | |
| miR-E | sh.Mcl1.1792 | ctcgagaaggtatattgctgttgaca gtgagcgaaacagcctcgatttttaa | 41 |
| | | gaatagtgaagccacagatgtattct taaaaatcgaggctgttctgcctact | |
| | | gcctcggacttcaaggggctagaatt c | |
| miR-E | sh.Mcl1.2018 | ctcgagaaggtatattgctgttgaca gtgagcgcggactggttatagattta | 42 |
| | | taatagtgaagccacagatgtattat aaatctataaccagtccatgcctact | |
| | | gcctcggacttcaaggggctagaatt c | |

TABLE 2
Additional shRNA sequences (SEQ ID NOS 43-61,
respectively, in order of appearance)

BRD4.602 (human)
TGCTGTTGACAGTGAGCGACAGGACTTCAACACTATGTTTTAGTGAAGCCACAGATGTAAAACATAGTGTTGAAGT
CCTGGTGCCTACTGCCTCGGA
BRD4.1817 (human)
TGCTGTTGACAGTGAGCGACAGCAGAACAAACCAAAGAAATAGTGAAGCCACAGATGTATTTCTTTGGTTTGTTCT
GCTGGTGCCTACTGCCTCGGA
BRD4.1838 (human)
TGCTGTTGACAGTGAGCGCAAGGAGAAAGACAAGAAGGAATAGTGAAGCCACAGATGTATTCCTTCTTGTCTTTCT
CCTTTTGCCTACTGCCTCGGA
Dnmt3a.16 (mouse)
TGCTGTTGACAGTGAGCGACAAGGTAATTGCAGTAATGAATAGTGAAGCCACAGATGTATTCATTACTGCAATTAC
CTTGGTGCCTACTGCCTCGGA
Dnmt3a.733 (mouse)
TGCTGTTGACAGTGAGCGACAGAAAGAGCACAACAGAGAATAGTGAAGCCACAGATGTATTCTCTGTTGTGCTCTT
TCTGGTGCCTACTGCCTCGGA
Dnmt3a.745 (mouse)
TGCTGTTGACAGTGAGCGAAACAGAGAAACCTAAGGTCAATAGTGAAGCCACAGATGTATTGACCTTAGGTTTCTC
TGTTGTGCCTACTGCCTCGGA
Dnmt3a.1177 (mouse)
TGCTGTTGACAGTGAGCGCCCAGATGTTCTTTGCCAATAATAGTGAAGCCACAGATGTATTATTGGCAAAGAACAT
CTGGATGCCTACTGCCTCGGA
Dnmt3a.1425 (mouse)
TGCTGTTGACAGTGAGCGAGCAGCGTCACACAGAAGCATATAGTGAAGCCACAGATGTATATGCTTCTGTGTGACG
CTGCGTGCCTACTGCCTCGGA
Dnmt3a.1546 (mouse)
TGCTGTTGACAGTGAGCGACCGCCTCTTCTTTGAGTTCTATAGTGAAGCCACAGATGTATAGAACTCAAAGAAGAG
GCGGCTGCCTACTGCCTCGGA
Dnmt3a.1691 (mouse)
TGCTGTTGACAGTGAGCGACCCGTGATGATTGACGCCAAATAGTGAAGCCACAGATGTATTTGGCGTCAATCATCA
CGGGGTGCCTACTGCCTCGGA
Dnmt3a.1777 (mouse)
TGCTGTTGACAGTGAGCGCGGCATCCACTGTGAATGATAATAGTGAAGCCACAGATGTATTATCATTCACAGTGGA
TGCCATGCCTACTGCCTCGGA
Dnmt3a.2338 (mouse)
TGCTGTTGACAGTGAGCGACTGGTGCTAATTCCTCTCATATAGTGAAGCCACAGATGTATATGAGAGGAATTAGCA
CCAGCTGCCTACTGCCTCGGA
Dnmt3a.4173 (mouse)
TGCTGTTGACAGTGAGCGCTAGCTGTTGAAGAAATATTAATAGTGAAGCCACAGATGTATTAATATTTCTTCAACA
GCTATTGCCTACTGCCTCGGA
Dnmt3a.4575 (mouse)
TGCTGTTGACAGTGAGCGCAAGGTGAAGTTGTCTCGTTTATAGTGAAGCCACAGATGTATAAACGAGACAACTTCA
CCTTATGCCTACTGCCTCGGA
Luci.1309 (Firefly Luciferase.control)
TGCTGTTGACAGTGAGCGCCCGCCTGAAGTCTCTGATTAATAGTGAAGCCACAGATGTATTAATCAGAGACTTCAG
GCGGTTGCCTACTGCCTCGGA
MYB.721 (human)
TGCTGTTGACAGTGAGCGACTGGACGAACTGATAATGCTATAGTGAAGCCACAGATGTATAGCATTATCAGTTCGT
CCAGGTGCCTACTGCCTCGGA
MYB.2487 (human)
TGCTGTTGACAGTGAGCGCAAGAAACTTGGTGTTAGGTAATAGTGAAGCCACAGATGTATTACCTAACACCAAGTT
TCTTTTGCCTACTGCCTCGGA
MYC.1834 (human & mouse)
TGCTGTTGACAGTGAGCGCACGACGAGAACAGTTGAAACATAGTGAAGCCACAGATGTATGTTTCAACTGTTCTCG
TCGTTTGCCTACTGCCTCGGA
RPA3.1401 (human)
TGCTGTTGACAGTGAGCGACCACCATCTTGTGTACATCTTTAGTGAAGCCACAGATGTAAAGATGTACACAAGATG
GTGGCTGCCTACTGCCTCGGA

1. A modified miRNA molecule consisting of a primary miRNA (pri-miRNA) molecule for producing an artificial shRNA molecule in a host cell, said pri-miRNA comprising
a stem-loop structure comprising a stem portion comprising a sequence encoding the artificial shRNA molecule, a loop portion at one end of the stem portion, and a Drosha cleavage site at the other end of the stem portion;
a 3′-flanking sequence comprising a region comprising a-sequence 5′ DC*NNC-3′ (SEQ ID NO: 62), wherein N consists of A, G, U, or C, D consists of A, G or U, and wherein the C* is located 16-20 nt from the Drosha cleavage site; and
a 5′-flanking sequence;
wherein the 3′ flanking sequence and the 5′ flanking sequence are attached to the Drosha cleavage site wherein the pri-miRNA converts to the artificial shRNA in the host cell, wherein the artificial shRNA molecule consists of a guide and a passenger strand and wherein the guide strand comprises a sequence that is complementary to a target mRNA sequence.

2. The modified miRNA molecule of claim 1, wherein the region of the 3′-flanking sequence consists of the sequence 5′-ACUUCAA-3′ (SEQ ID NO: 63), 5′-GCUUCGA-3′ (SEQ ID NO: 64), 5′-UCUUCUG-3′ (SEQ ID NO: 65), 5′-GACUUCAA-3′ (SEQ ID NO: 101), 5′-UACUUCAA-3′ (SEQ ID NO:102), 5′-GACGUCAA-3′ (SEQ ID NO:103), 5′-GACUGCAA-3′ (SEQ ID NO:104), 5′-GACUUCCA-3′ (SEQ ID NO:105), or 5′-GACUUCAC-3′ (SEQ ID NO:106).

3. The modified miRNA molecule of claim 2, wherein the 3′-flanking sequence or the 5′-flanking sequence comprises a modified sequence that encodes a recognition site for a restriction enzyme.

4. The modified miRNA molecule of claim 3, wherein the recognition site consists of a sequence recognized by EcoRI or XhoI.

5. The modified miRNA molecule of claim 3, wherein the modified sequence that encodes the recognition site for the restriction enzyme consists of 5′-GAAUUC-3′ (SEQ ID NO: 66) and the complementary strand consists of the sequence 5′-GACUUC-3′ (SEQ ID NO: 67).

6. The modified miRNA_molecule of claim 2, wherein the 3′-flanking sequence or the 5′-flanking sequence or both the 3′- and 5′ flanking sequences comprise elements that preserve secondary structure of the native pri-miRNA molecule.

7. The modified miRNA molecule of claim 2, wherein the double-stranded stem portion does not include a bulge or a mismatch between the guide and passenger strand.

8. The modified miRNA molecule of claim 2, wherein the modified miRNA molecule comprises a pri-miR-30 molecule.

9. The modified miRNA molecule of claim 2, wherein the miRNA molecule comprises a pri-miR-15/16 or a miR-22 molecule.

10. The modified miRNA molecule of claim 2, wherein the pri-miRNA molecule is efficiently processed by the host cell to the shRNA molecule.

11. The modified miRNA molecule of claim 10, wherein the shRNA molecule is efficiently processed by the host cell to a siRNA molecule consisting of the guide and the passenger strands.

12. The modified miRNA molecule of claim 10, wherein the pri-miRNA molecule is efficiently processed to the shRNA molecule in the host cell nucleus.

13. The modified miRNA molecule of claim 10, wherein the shRNA molecule is efficiently processed by Dicer in the host cell cytoplasm.

14. A nucleic acid construct encoding the modified miRNA molecule of claim 2.

15. The nucleic acid construct of claim 14 comprising a Pol II promoter.

16. The nucleic acid construct of claim 15, wherein the Pol II promoter is a constitutive promoter, an inducible promoter, a ubiquitous promoter, a tissue-specific promoter, a cell-type-specific promoter, and/or a developmental stage-specific promoter.

17. The nucleic acid construct of claim 16, wherein the Pol II promoter comprises a sequence of tightly regulatable TET system a promoter from cytomegalovirus.

18. The nucleic acid construct of claim 14, further comprising at least one selectable marker.

19. The nucleic acid construct of claim 14, further comprising a reporter gene.

20. The nucleic acid construct of claim 14, further comprising a Pol III promoter upstream of a sequence encoding the modified miRNA molecule.
